(19) United States
(12) Patent Application Publication
(10) Pub. No.: US 2008/0184495 A1
(43) Pub. Date: Aug. 7, 2008

(54) Dye composition comprising a reactive silicone and a fluorescent dye, and dyeing process using the composition

(76) Inventors: Gaelle Brun, Paris (FR); Luc Gourlaouen, Asnieres (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 12/004,072

(22) Filed: Dec. 20, 2007

Related U.S. Application Data
(60) Provisional application No. 60/878,457, filed on Jan. 4, 2007.

(30) Foreign Application Priority Data
Dec. 20, 2007 (FR) 0655719

(51) Int. Cl. A61K 8/49; A61Q 5/10

(52) U.S. Cl. 8/425

(57) ABSTRACT

The present invention relates to a process for dyeing keratin fibers, which consists in applying to the fibers a dye composition comprising: i) at least one compound X; ii) at least one compound Y, wherein compounds X and Y, when placed in contact with each other, react together via a hydrosilylation reaction optionally in the presence of a hydrosilylation catalyst; a condensation reaction; or a crosslinking reaction in the presence of at least one peroxide; and iii) at least one fluorescent dye.

The coloration obtained after applying the composition to the hair shows good resistance to various attacking factors. Moreover, the hairs remain perfectly individualized and may be easily styled.

This application claims benefit of U.S. Provisional Application No. 60/878,457, filed Jan. 4, 2007, the contents of which are incorporated herein by reference. This application also claims benefit of priority under 35 U.S.C. § 119 to French Patent Application No. FR 0655719, filed Dec. 20, 2006, the contents of which are also incorporated herein by reference.

The present disclosure relates to a dye composition comprising at least one reactive silicone and at least one fluorescent dye that is useful for dyeing keratin fibers.

It has been sought for a long time to color the hair and in particular to mask grey hair. Several techniques have been developed to do this.

It is known practice to dye keratin fibers, for example human keratin fibers such as the hair, with dye compositions comprising direct dyes. The standard dyes that are used are, for example, dyes of the nitrobenzene, anthraquinone, nitropyridine, azo, xanthene, acridine, azine or triarylmethane type or natural dyes. These dyes may be nonionic, anionic, cationic or amphoteric. These dyes, which are colored and coloring molecules with affinity for keratin fibers, may be applied for the time necessary to obtain the desired coloration, and are then rinsed out. The resulting colorations are particularly chromatic, but may, however, be temporary or semi-permanent since the nature of the interactions that bind the direct dyes to the keratin fibers, and their desorption from the surface and/or the core of the fibers, are responsible for their typically weak dyeing power and poor fastness with respect to washing or perspiration.

It is also known practice to dye keratin fibers permanently by oxidation dyeing. This dyeing technique comprises applying to keratin fibers a composition comprising dye precursors such as oxidation bases and couplers. Under the action of an oxidizing agent, these precursors may form at least one colored species in the hair. The variety of molecules used as oxidation bases and couplers allows a wide range of colors to be obtained. The colorations resulting therefrom are permanent, strong and/or resistant to external agents, such as light, bad weather, washing, perspiration and rubbing. However, this type of dyeing may lead to degradation of the fiber due to the use of an oxidizing agent.

It would therefore be desirable to develop novel direct dye compositions to obtain varied shades, for example pastel shades, which show good fastness, such as with respect to external agents such as light, shampoo and sweat, while at the same time preserving the quality of the keratin fibers. Moreover, it would be desirable to develop dye compositions that can produce colorations whose fastness is close to that of oxidation dyeing, without at least one of the drawbacks associated with the presence of an oxidizing agent.

Moreover, the dyeing of keratin fibers using standard direct dyes does not make it possible to appreciably lighten keratin fibers. Indeed, there is considerable demand for lightening of the color of keratin fibers, for example of dark fibers, towards lighter shades, by possibly modifying the shade of these fibers.

Conventionally, to obtain a lighter coloration a chemical bleaching process is used. This process comprises treating keratin fibers with a strong oxidizing system, generally comprising hydrogen peroxide optionally combined with persalts, usually in alkaline medium.

As has been seen previously, the bleaching system may have the drawback of degrading keratin materials, such as keratin fibers, for example human keratin fibers such as the hair, and of impairing their cosmetic properties. Specifically, the fibers may have a tendency to become coarse, more difficult to disentangle and/or more brittle. Finally, the lightening or bleaching of keratin fibers with oxidizing agents may be incompatible with treatments for modifying the shape of the fibers, for example in relaxing treatments.

Another lightening technique comprises applying to dark hair fluorescent direct dyes. This technique described, for example, in French Patent No. 2 830 189 and in International Patent Application WO 2004/091473 makes it possible to respect the quality of the keratin fiber during the treatment, but the fluorescent dyes used do not always show satisfactory resistance to shampoo.

All the systems described using pigments or fluorescent dye, therefore, do not always show satisfactory remanence with respect to external attack, sebum, perspiration, bad weather and/or successive shampooing.

However, the present inventors have found, unexpectedly, that it may be possible, with the present disclosure, to solve the technical problem of remanence without impairing the cosmetic qualities of the fibers or the integrity of the fibers, and/or without reducing the dyeing or lightening properties originating from the pigments or the fluorescent dyes.

Thus, disclosed herein is a process for dyeing keratin fibers, which comprises applying to said fibers a dye composition comprising: i) at least one compound X; ii) at least one compound Y, and iii) at least one fluorescent dye, wherein at least one of compounds X or Y is a silicone compound, and wherein said compounds X and Y react together via a hydrosilylation reaction, optionally in the presence of a hydrosilylation catalyst; or a condensation reaction or a crosslinking reaction in the presence of a peroxide, when they are placed in contact with each other.

Also disclosed herein is a dye composition comprising: i) at least one compound X; ii) at least one compound Y, and iii) at least one fluorescent dye, wherein at least one of compounds X or Y is a silicone compound, and wherein said compounds X and Y react or are capable of reacting together via a hydrosilylation reaction, optionally in the presence of a hydrosilylation catalyst; or a condensation reaction or a crosslinking reaction in the presence of a peroxide, when they are placed in contact with each other.

Further disclosed herein is the use of the mixture of X and Y optionally in the presence of at least one hydrosilylation catalyst or at least one peroxide; and at least one fluorescent dye; for dyeing keratin fibers and lightening dark keratin fibers.

Still further disclosed herein is a dyeing kit or device comprising at least two compositions (A) and (B) packaged separately, the kit comprising i) at least one compound X, ii) at least one compound Y, wherein composition (A) and/or (B) comprises at least one fluorescent dye; and iii) optionally at least one catalyst or at least one peroxide; wherein the at least one catalyst, when it is present, and the at least one peroxide are not simultaneously present in the same composition; said compounds X and Y react or are capable of reacting together via a hydrosilylation reaction, or via a condensation reaction, or via a crosslinking reaction in the presence of a peroxide, when they are placed in contact with each other.

In at least one embodiment, the composition disclosed herein makes it possible to improve the visibility of the coloration, such as on dark hair. In a further embodiment, in the case of dark keratin fibers, a very visible coloration may be obtained without it being necessary to lighten or bleach the keratin fibers and consequently without any physical degradation of the keratin fibers. In another embodiment, the composition disclosed herein may make it possible to obtain optical lightening effects on dark hair, such as hair with a tone depth of less than or equal to 6, for example less than or equal to 4.

By means of the choice of the fluorescent compound, the composition also may make it possible to obtain colorations with special effects under the effect of light rich in UV radiation, such as the lighting used, for example, in night clubs, for example black light.

Furthermore, this coloration may show good resistance to various attacking factors to which the hair may be subjected, such as shampoo, rubbing, light, bad weather, sweat and/or permanent reshaping. In at least one embodiment, the coloration shows good resistance with respect to shampooing. In a further embodiment, the hair shows good cosmetic properties, for example the hairs may remain perfectly individualized and may be easily styled.

### I) Compounds X and Y

As used herein, the term “silicone compound” is intended to mean a compound comprising at least two organosiloxane units. According to one embodiment, the at least one compound X and the at least one compound Y are silicone-based. In another embodiment, the at least one compound X and the at least one compound Y may be amino or non-amino compounds. In yet another embodiment, they may comprise polar groups, which may be chosen from the following groups: —COOH, —COO⁻, —COO—, —OH, —NH₂, —NH—, —NR—, —SO₃H, —SO₃⁻, —OCH₂CH₂—, —O—CH₂CH₂CH₂—, —O—CH₂CH(CH₃)—, —NR₃⁺, —SH, —NO₂, I, Cl, Br, —CN, —PO₄³⁻, —CONH—, —CONR—, —CONH₂, —CSNH—, —SO₂—, —SO—, —SO₂NH—, —NHCO—, —NHSO₂—, —NHCOO—, —OCONH—, —NHCSO— and —OCSNH— wherein R represents an alkyl group.

According to another embodiment, at least one of the compounds X and Y is a polymer whose main chain is predominantly formed from organosiloxane units.

Among the silicone compounds mentioned below, some may have both film-forming properties and adhesive properties depending, for example, on their proportion of silicone or on whether they are used as a mixture with a particular additive. It is consequently possible to modify the film-forming properties or the adhesive properties of such compounds according to the intended use, such as for the reactive elastomeric silicones said to be “room-temperature vulcanizable”.

In at least one embodiment, compounds X and Y may react together at a temperature ranging from room temperature to 180° C. In a further embodiment, compounds X and Y may react together at room temperature (20±5° C.) and atmospheric pressure, and in one embodiment in the presence of at least one catalyst, via a hydrosilylation reaction or a condensation reaction, or a crosslinking reaction in the presence of at least one peroxide.

### 1—Compounds X and Y-Hydrosilylation Reaction

According to one embodiment, compounds X and Y react (or are capable of reacting) via hydrosilylation, this reaction being represented schematically in simple terms as follows:

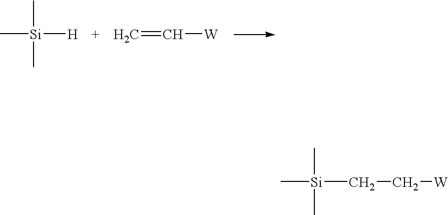

wherein W represents a carbon-based and/or silicone chain comprising at least one unsaturated aliphatic group.

In this case, compound X may be chosen from silicone compounds comprising at least two unsaturated aliphatic groups. For example, compound X may comprise a silicone main chain whose unsaturated aliphatic groups are pendent on the main chain (side group) or located at the ends of the main chain of the compound (end group). In the rest of the description herein, these particular compounds will be referred to as “polyorganosiloxanes comprising unsaturated aliphatic groups.”

According to one embodiment, the at least one compound X is chosen from polyorganosiloxanes comprising at least two unsaturated aliphatic groups, for example two or three vinyl or allylic groups, each bonded to a silicon atom.

According to one embodiment, the at least one compound X is chosen from polyorganosiloxanes comprising siloxane units of formula:

$${R}_{m} {R}^{′} {SiO}_{\frac{( 3 - m )}{2}} \qquad ( I )$$

wherein:

R is chosen from linear or cyclic monovalent hydrocarbon-based groups comprising from 1 to 30 carbon atoms, for example from 1 to 20 or from 1 to 10 carbon atoms, for instance a short-chain alkyl radical comprising, for example, from 1 to 10 carbon atoms, such as a methyl radical, or alternatively a phenyl group,

m is equal to 1 or 2, and

R′ is chosen from:
unsaturated aliphatic hydrocarbon-based groups comprising from 2 to 10 carbon atoms, for example from 2 to 5 carbon atoms, for instance vinyl groups or groups of the formula —R″—CH═CHR′″ wherein R″ is a divalent aliphatic hydrocarbon-based chain comprising from 1 to 8 carbon atoms, bonded to the silicon atom and R′″ is chosen from hydrogen atoms and alkyl radicals comprising from 1 to 4 carbon atoms; for example, R′ may be chosen from vinyl and allylic groups and mixtures thereof; and
unsaturated cyclic hydrocarbon-based groups comprising from 5 to 8 carbon atoms, for instance cyclohexenyl groups.

In at least one embodiment, R′ is an unsaturated aliphatic hydrocarbon-based group, for example a vinyl group.

According to one embodiment, the polyorganosiloxane may also comprise at least one unit of formula:

$${R}_{n} {SiO}_{\frac{( 4 - n )}{2}} \qquad ( II )$$

wherein R is defined above, and n is equal to 1, 2 or 3.

In one embodiment, compound X may be a silicone resin comprising at least two ethylenic unsaturations, wherein said resin reacts or is capable of reacting with compound Y via hydrosilylation. Examples of silicone resins include, but are not limited to, resins of MQ or MT type comprising at least one —CH═CH₂ unsaturated reactive end.

In at least one embodiment, these resins are crosslinked organosiloxane polymers.

As used herein, the nomenclature of silicone resins is represented by “MDTQ”, wherein the resin is described as a function of the various siloxane monomer units it comprises, and wherein each of the letters M, D, T and Q characterizes a type of unit.

As used herein, the letter M represents the monofunctional unit of formula (CH₃)₃SiO_{1/2}, wherein the silicon atom is bonded to only one oxygen atom in the polymer comprising this unit.

As used herein, the letter D represents a difunctional unit (CH₃)₂SiO_{2/2}, wherein the silicon atom is bonded to two oxygen atoms.

As used herein, the letter T represents a trifunctional unit of formula (CH₃)₁SiO_{3/2}.

In the units M, D and T defined above, at least one of the methyl groups may be substituted with a group R other than a methyl group, such as a hydrocarbon-based radical, for example alkyl, comprising from 2 to 10 carbon atoms, or a phenyl group, or alternatively a hydroxyl group.

As used herein, the letter Q represents a tetrafunctional unit SiO_{4/2} in which the silicon atom is bonded to four hydrogen atoms, which are themselves bonded to the rest of the polymer. Examples of such resins include, but are not limited to, MT silicone resins such as poly(phenylvinylsilsesquioxane), for instance the product sold under the reference SST-3PV1 by the company Gelest.

In at least one embodiment, the compounds X comprise from 0.01% to 1% by weight of unsaturated aliphatic groups relative to the total weight of the compound.

In at least one embodiment, compound X is chosen from polyorgano-polysiloxanes, for example those comprising at least one siloxane unit (I) and optionally (II) as described above.

Compound Y then comprises at least two free Si—H groups (hydrogenosilane groups).

In at least one embodiment, compound Y may be chosen from organo-siloxanes comprising at least one alkylhydrogenosiloxane unit having the following formula:

$${R}_{p} {H SiO}_{\frac{( 3 - p )}{2}} \qquad ( III )$$

wherein:

R is chosen from linear or cyclic monovalent hydrocarbon-based groups comprising from 1 to 30 carbon atoms, for instance an alkyl radical comprising from 1 to 30 carbon atoms, for example from 1 to 20 or from 1 to 10 carbon atoms, such as a methyl radical, or alternatively a phenyl group, and p is equal to 1 or 2. In one embodiment, R is a hydrocarbon-based group, for example methyl.

These organosiloxane compounds Y comprising alkylhydrogenosiloxane units may also comprise units of formula:

$${R}_{n} {SiO}_{\frac{( 4 - n )}{2}} \qquad ( II )$$

as defined above.

In at least one embodiment, compound Y may be a silicone resin comprising at least one unit chosen from the units M, D, T and Q as defined above and comprising at least one Si—H group, such as the poly(methylhydridosilsesquioxanes) sold under the reference SST-3 MH1.1 by the company Gelest.

In at least one embodiment, these organosiloxane compounds Y comprise Si—H groups in an amount ranging from 0.5% to 2.5% by weight relative to the total weight of the compound.

In at least one embodiment, the radicals R represent a methyl group in formulae (I), (II) and (III) above.

In at least one embodiment, the organosiloxane(s) Y comprise end group(s) of formula (CH₃)₃SiO_{1/2}.

In another embodiment, the organosiloxane(s) Y comprise at least two alkylhydrogenosiloxane units of formula (H₃C)(H)SiO and optionally comprise (H₃C)₂SiO units.

Such organosiloxane compounds Y comprising hydrogenosilane groups are described, for example, in European Patent No. 0 465 744.

According to one embodiment, compound X is chosen from organic oligomers or polymers (as used herein, the term “organic” means compounds whose main chain is not silicone-based, for example compounds comprising no silicon atoms) and from organic/silicone hybrid polymers or oligomers, the said oligomers or polymers bearing at least 2 reactive unsaturated aliphatic groups, and compound Y is chosen from hydrogenosiloxanes mentioned above.

Compound X, of organic nature, may then be chosen from vinyl or (meth)acrylic polymers or oligomers, polyesters, polyurethanes and/or polyureas, polyethers, perfluoropolyethers, polyolefins such as polybutene or polyisobutylene, dendrimers and organic hyperbranched polymers, and mixtures thereof.

In a further embodiment, the organic polymer or the organic part of the hybrid polymer may be chosen from the following polymers:

a) ethylenically unsaturated polyesters: This is a group of polymers of polyester type comprising at least two ethylenic double bonds, randomly distributed in the main polymer chain, wherein the unsaturated polyesters are obtained by polycondensation of a mixture:
of linear or branched aliphatic or cycloaliphatic dicarboxylic acids, which in at least one embodiment comprise from 3 to 50 carbon atoms, for example from 3 to 20 or from 3 to 10 carbon atoms, such as adipic acid or sebacic acid; and/or of aromatic dicarboxylic acids, which in at least one embodiment comprise from 8 to 50 carbon atoms, for example from 8 to 20 or from 8 to 14 carbon atoms, such as phthalic acids, terephthalic acid, and/or of dicarboxylic acids derived from ethylenically unsaturated fatty acid dimers such as the oleic or linoleic acid dimers described in European patent application EP-A-959 066 (paragraph [0021]) sold under the names Pripol® by the company Uniqema or Empol® by the company Henkel, wherein all said diacids are free of polymerizable ethylenic double bonds;
of linear or branched aliphatic or cycloaliphatic diols, which in one embodiment comprise from 2 to 50 carbon atoms, for example from 2 to 20 or from 2 to 10 carbon atoms, such as ethylene glycol, diethylene glycol, propylene glycol, 1,4-butanediol or cyclohexanedimethanol; of aromatic diols comprising from 6 to 50 carbon atoms, for example from 6 to 20 or from 6 to 15 carbon atoms, such as bisphenol A and bisphenol B; and/or of diol dimers obtained from the reduction of fatty acid dimers as defined above; and
of at least one dicarboxylic acid or anhydride thereof comprising at least one polymerizable ethylenic double bond and comprising from 3 to 50 carbon atoms, for example from 3 to 20 or from 3 to 10 carbon atoms, such as maleic acid, fumaric acid or itaconic acid; >

b) polyesters comprising at least one (meth)acrylate side group and/or end group: This is a group of polymers of polyester type obtained by polycondensation of a mixture:
of linear or branched aliphatic or cycloaliphatic dicarboxylic acids, which in one embodiment comprise from 3 to 50 carbon atoms, for example from 3 to 20 or from 3 to 10 carbon atoms, such as adipic acid or sebacic acid; of aromatic dicarboxylic acids, which in one embodiment comprise from 8 to 50 carbon atoms, for example from 8 to 20 or from 8 to 14 carbon atoms, such as phthalic acids, or terephthalic acid; and/or of dicarboxylic acids derived from ethylenically unsaturated fatty acid dimers such as the oleic acid or linoleic acid dimers described in European patent application EP-A-959 066 (paragraph [0021]) sold under the names Pripol® by the company Uniqema or Empol® by the company Henkel, wherein all said diacids are free of polymerizable ethylenic double bonds;
of linear or branched aliphatic or cycloaliphatic diols, which in one embodiment comprise from 2 to 50 carbon atoms, for example from 2 to 20 or from 2 to 10 carbon atoms, such as ethylene glycol, diethylene glycol, propylene glycol, 1,4-butanediol or cyclohexanedimethanol; of aromatic diols comprising from 6 to 50 carbon atoms, for example from 6 to 20 or from 6 to 15 carbon atoms, such as bisphenol A and bisphenol B; and
of at least one ester of (meth)acrylic acid and of a diol or polyol comprising from 2 to 20 carbon atoms, for example from 2 to 6 carbon atoms, such as 2-hydroxyethyl (meth)acrylate, 2-hydroxypropyl(meth)acrylate or glycerol methacrylate.

These polyesters differ from those described above in point a) by the fact that the ethylenic double bonds are not located in the main chain but on side groups or at the end of the chains; and wherein the ethylenic double bonds are those of the at least one (meth)acrylate group present in the polymer.

Such polyesters are sold, for example, by the company UCB under the names Ebecryl® (Ebecryl® 450: molar mass 1600, on average 6 acrylate functions per molecule, Ebecryl® 652: molar mass 1500, on average 6 acrylate functions per molecule, Ebecryl® 800: molar mass 780, on average 4 acrylate functions per molecule, Ebecryl® 810: molar mass 1000, on average 4 acrylate functions per molecule, Ebecryl® 50 000: molar mass 1500, on average 6 acrylate functions per molecule);

c) polyurethanes and/or polyureas comprising at least one (meth)acrylate group, obtained by polycondensation
of aliphatic, cycloaliphatic and/or aromatic diisocyanates, triisocyanates and/or polyisocyanates, which in one embodiment comprise from 4 to 50 carbon atoms, for example from 4 to 30 carbon atoms, such as hexamethylene diisocyanate, isophorone diisocyanate, toluene diisocyanate, diphenylmethane diisocyanate or isocyanurates of formula

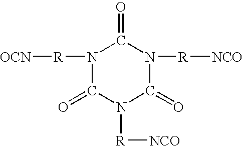

resulting from the trimerization of 3 molecules of diisocyanates OCN—R—CNO, wherein R is chosen from linear, branched and cyclic hydrocarbon-based radicals comprising from 2 to 30 carbon atoms;
of polyols, and in one embodiment of diols, free of polymerizable ethylenic unsaturations, such as 1,4-butanediol, ethylene glycol or trimethylolpropane, and/or aliphatic, cycloaliphatic and/or aromatic polyamines, for example diamines, such as those comprising from 3 to 50 carbon atoms, such as ethylenediamine or hexamethylenediamine; and
of at least one ester of (meth)acrylic acid and of a diol or polyol comprising from 2 to 20 carbon atoms, for example from 2 to 6 carbon atoms, such as 2-hydroxyethyl (meth)acrylate, 2-hydroxypropyl(meth)acrylate or glycerol methacrylate.

Such polyurethanes/polyureas comprising at least one acrylate group are sold, for example, under the name SR 368 (tris(2-hydroxyethyl) isocyanurate-triacrylate) or Craynor® 435 by the company Cray Valley, or under the name Ebecry® by the company UCB (Ebecryl® 210: molecular mass 1500, 2 acrylate functions per molecule, Ebecryl® 230: molecular mass 5000, 2 acrylate functions per molecule, Ebecryl® 270: molecular mass 1500, 2 acrylate functions per molecule, Ebecryl® 8402: molecular mass 1000, 2 acrylate functions per molecule, Ebecryl® 8804: molecular mass 1300, 2 acrylate functions per molecule, Ebecryl® 220: molecular mass 1000, 6 acrylate functions per molecule, Ebecryl® 2220: molecular mass 1200, 6 acrylate functions per molecule, Ebecryl® 1290: molecular mass 1000, 6 acrylate functions per molecule, Ebecryl® 800: molecular mass 800, 6 acrylate functions per molecule);

Non-limiting mention may also be made of the water-soluble aliphatic diacrylate polyurethanes sold under the names Ebecryl® 2000, Ebecryl® 2001 and Ebecryl® 2002, and the diacrylate polyurethanes in aqueous dispersion sold under the trade names IRR® 390, IRR® 400, IRR® 422 and IRR® 424 by the company UCB;

d) polyethers comprising at least one (meth)acrylate group obtained by esterification, with (meth)acrylic acid, of the hydroxyl end groups of C₁₋₄ alkylene glycol homopolymers or copolymers, such as polyethylene glycol, polypropylene glycol, copolymers of ethylene oxide and of propylene oxide, which in one embodiment has a weight-average molecular mass of less than 10,000, and polyethoxylated or polypropoxylated trimethylolpropane.

Polyoxyethylene di(meth)acrylates of suitable molar mass include, but are not limited to, those sold, for example, under the names SR 259, SR 344, SR 610, SR 210, SR 603 and SR 252 by the company Cray Valley or under the name Ebecryl® 11 by UCB. Polyethoxylated trimethylolpropane triacrylates useful herein include, but are not limited to, those sold under the names SR 454, SR 498, SR 502, SR 9035 and SR 415 by the company Cray Valley or under the name Ebecryl® 160 by the company UCB. Polypropoxylated trimethylolpropane triacrylates useful herein include, but are not limited to, those sold, for example, under the names SR 492 and SR 501 by the company Cray Valley;

e) epoxyacrylates obtained by reaction between
at least one diepoxide chosen, for example, from:
(i) bisphenol A diglycidyl ethers;
(ii) diepoxy resins resulting from the reaction between bisphenol A diglycidyl ether and epichlorohydrin;
(iii) epoxy ester resins comprising α,ω-diepoxy end groups resulting from the condensation of a dicarboxylic acid comprising from 3 to 50 carbon atoms with a stoichiometric excess of (i) and/or (ii);
(iv) epoxy ether resins comprising α,ω-diepoxy end groups resulting from the condensation of a diol comprising from 3 to 50 carbon atoms with a stoichiometric excess of (i) and/or (ii);
(v) natural or synthetic oils comprising at least 2 epoxide groups, such as epoxidized soybean oil, epoxidized linseed oil or epoxidized vernonia oil;
(vi) phenol-formaldehyde polycondensates (for example Novolac® resin), the end groups and/or side groups of which have been epoxidized, and
at least one carboxylic acid or polycarboxylic acid comprising at least one ethylenic double bond in the α,β-position relative to the carboxylic group, for instance (meth)acrylic acid or crotonic acid or esters of (meth)acrylic acid and of at least one diol or polyol comprising from 2 to 20 carbon atoms, for example from 2 to 6 carbon atoms, such as 2-hydroxyethyl(meth)acrylate,

Such polymers are sold, for example, under the names SR 349, SR 601, CD 541, SR 602, SR 9036, SR 348, CD 540, SR 480 and CD 9038 by the company Cray Valley, under the names Ebecryl® 600, Ebecryl® 609, Ebecryl® 150, Ebecryl® 860 and Ebecryl® 3702 by the company UCB and under the names Photomer® 3005 and Photomer® 3082 by the company Henkel;

f) poly(C₁₋₅₀ alkyl(meth)acrylates), wherein said alkyl is linear, branched or cyclic, comprising at least two functions comprising an ethylenic double bond borne by the hydrocarbon-based side chains and/or end chains,

Such copolymers are sold, for example, under the names IRR® 375, OTA® 480 and Ebecryl® 2047 by the company UCB;

g) polyolefins such as polybutene or polyisobutylene;

h) perfluoropolyethers comprising at least one acrylate group obtained by esterification, for example with (meth)acrylic acid, of perfluoropolyethers comprising hydroxyl side groups and/or end groups,

Such α,ω-diol perfluoropolyethers are described, for example, in EP-A-1 057 849 and are sold by the company Ausimont under the name Fomblin® Z Diol.

i) hyperbranched dendrimers and polymers bearing (meth)acrylate or (meth)acrylamide end groups obtained, respectively, by esterification or amidation of hyperbranched dendrimers and polymers comprising at least one hydroxyl or amino end function, with (meth)acrylic acid.

As used herein, “dendrimers” (from the Greek dendron=tree) are “arborescent”, i.e. highly branched, polymer molecules, such as those invented by D. A. Tomalia and his team at the start of the 1990s (Donald A. Tomalia et al., Angewandte Chemie, Int. Engl. Ed., Vol. 29, No. 2, pages 138-175). These are structures constructed about a central unit that is generally polyvalent. About this central unit are linked, in a fully determined structure, branched chain-extending units, thus giving rise to monodispersed symmetrical macromolecules having a well-defined chemical and stereochemical structure. Dendrimers of polyamidoamine type are sold, for example, under the name Starburst® by the company Dendritech.

As used herein, “hyperbranched polymers” are polycondensates, generally of polyester, polyamide or polyethyleneamine type, obtained from multifunctional monomers, which have an arborescent structure similar to that of dendrimers but are much less regular than dendrimers (see, for example, international patent applications WO-A-93/17060 and WO 96/12754).

Examples of hyperbranched polyesters include those sold by the company Perstorp under the name Boltorn®. Examples of hyperbranched polyethyleneamines include those sold under the name Comburst® from the company Dendritech. Examples of hyperbranched poly(esteramides) comprising hydroxyl end groups include those sold by the company DSM under the name Hybrane®.

The hyperbranched dendrimers and polymers esterified or amidated with acrylic acid and/or methacrylic acid disclosed herein may be distinguished from the polymers described in points a) to h) above, for example, by the very large number of ethylenic double bonds present. This high functionality, usually greater than 5, may make them particularly useful by allowing them to act as “crosslinking nodes”, i.e. sites of multiple crosslinking.

In at least one embodiment, the dendritic and hyperbranched polymers disclosed herein may thus be used in combination with at least one of the polymers and/or oligomers a) to h) above.

1a—Additional Reactive Compounds

According to at least one embodiment, the compositions comprising compound X and/or Y may also comprise an additional reactive compound such as:

organic or mineral particles comprising at their surface at least 2 unsaturated aliphatic groups: for example silicas surface-treated, for example, with silicone compounds comprising vinyl groups, for instance cyclotetramethyltetravinylsiloxane-treated silica; or

silazane compounds such as hexamethyldisilazane.

1b—Catalyst

In at least one embodiment, the hydrosilylation reaction may be performed in the presence of at least one catalyst that may be present in a composition containing the at least one compound X and/or in a composition containing at least one compound Y or in a separate composition. In one embodiment the catalyst may be platinum-based or tin-based.

Examples of catalysts include, but are not limited to, platinum-based catalysts deposited on a support of silica gel or charcoal powder (coal), platinum chloride, platinum salts and chloroplatinic acids.

In one embodiment, chloroplatinic acids in hexahydrate or anhydrous form, which are readily dispersible in organosilicone media are used. Mention may also be made of platinum complexes such as those based on chloroplatinic acid hexahydrate and on divinyltetramethyldisiloxane.

The catalyst may be present in one or the other of the compositions disclosed herein in an amount ranging from 0.0001% to 20% by weight relative to the total weight of the composition comprising it.

In at least one embodiment, at least one polymerization inhibitor, retardant or catalyst inhibitor may also be introduced into at least one composition of the disclosure, in order to increase the stability of the composition over time or to retard the polymerization. Non-limiting examples that may be mentioned include cyclic polymethylvinylsiloxanes, for example tetravinyltetramethylcyclotetrasiloxane, and acetylenic alcohols, which are in one embodiment volatile, such as methylisobutynol.

The presence of ionic salts, such as sodium acetate, in at least one of the first and second compositions may have an influence on the rate of polymerization of the compounds.

In one embodiment, compounds X and Y are chosen from silicone compounds which react or are capable of reacting via hydrosilylation; for example, compound X may be chosen from polyorganosiloxanes comprising units of formula (I) described above and compound Y may be chosen from organosiloxanes comprising alkylhydrogenosiloxane units of formula (III) described above. In one embodiment, compound X is a polydimethylsiloxane comprising at least one vinyl end group and compound Y is methylhydrogenosiloxane.

Examples of combinations of compounds X and Y that react via hydrosilylation include, but are not limited to, the following mixtures A and B prepared by Dow Corning:

### Mixture A:

| Ingredient (INCI name) | CAS No. | Content (%) | Function |
|---|---|---|---|
| Dimethylsiloxane, | 68083-19-2 | 55-95 | Polymer |
| Dimethylvinylsiloxy-terminated | | | |
| Silica Silylate | 68909-20-6 | 10-40 | Filler |
| 1,3-Diethenyl-1,1,3,3-Tetra- | 68478-92-2 | Trace | Catalyst |
| methyldisiloxane complexes | | | |
| Tetramethyldivinyldisiloxane | 2627-95-4 | 0.1-1 | Polymer |

### Mixture B:

| Ingredient (INCI name) | CAS No. | Content (%) | Function |
|---|---|---|---|
| Dimethylsiloxane, | 68083-19-2 | 55-95 | Polymer |
| Dimethylvinylsiloxy- | | | |
| terminated | | | |
| Silica Silylate | 68909-20-6 | 10-40 | Filler |
| Dimethyl, Methylhydrogen- | 68037-59-2 | 1-10 | Polymer |
| siloxane, trimethylsiloxy- | | | |
| terminated | | | |

### 2—Compounds X and Y-Condensation Reaction

In one embodiment, compounds X and Y react (or are capable of reacting) via condensation, either in the presence of water (hydrolysis) by reaction of two compounds bearing alkoxysilane groups, or via “direct” condensation by reaction of a compound bearing at least one alkoxysilane group and a compound bearing at least one silanol group or by reaction of two compounds, each bearing at least one silanol group.

When the condensation is performed in the presence of water, this water may, in one embodiment, be ambient moisture, sweat or the water provided by an external source, for example premoistening of the keratin fibers (for example with a mister).

In at least one embodiment, when the composition disclosed herein comprises compounds X and Y which react via condensation, then the at least one fluorescent dye is other than Rhodamine 101:

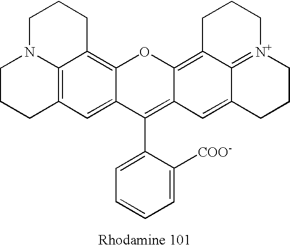

In this mode of reaction via condensation, compounds X and Y, which may be identical or different, may thus be chosen from silicone compounds whose main chain comprises at least two alkoxysilane groups and/or at least two silanol (Si—OH) groups, on the side and/or at the end of the chain.

In one embodiment, compounds X and/or Y are chosen from polyorganosiloxanes comprising at least two alkoxysilane groups. As used herein, the term “alkoxysilane group” means a group comprising at least one —Si—OR portion, wherein R is an alkyl group comprising from 1 to 6 carbon atoms.

In a further embodiment, compounds X and Y are chosen from poly-organosiloxanes comprising alkoxysilane end groups, for example those comprising at least 2 alkoxysilane end groups and/or trialkoxysilane end groups.

In at least one embodiment, the compounds X and/or Y predominantly comprise units of formula:

R⁹ₛSiO₍₄₋ₛ₎/2, (IV)

wherein R⁹ independently represents a radical chosen from alkyl groups comprising from 1 to 6 carbon atoms, phenyl and fluoroalkyl groups, and s is equal to 0, 1, 2 or 3. In a further embodiment, R⁹ independently represents an alkyl group comprising from 1 to 6 carbon atoms. Examples of such alkyl groups include, but are not limited to, methyl, propyl, butyl, and hexyl, and mixtures thereof. In one embodiment, the alkyl groups are chosen from methyl and ethyl. An exemplary fluoroalkyl group that may be mentioned is 3,3,3-trifluoropropyl.

According to at least one embodiment, compounds X and Y, which may be identical or different, are polyorganosiloxanes comprising units of formula:

(R⁹₂SiO₂)_{f}— (V)

wherein R⁹ is as described above, and in one embodiment R⁹ is a methyl radical, and f is such that the polymer has a viscosity at 25° C. ranging from 0.5 to 3000 Pa·s, for example ranging from 5 to 150 Pa·s. In another embodiment, f is a number ranging from 2 to 50,000, for example from 3 to 3000 or from 5 to 1000.

In one embodiment, the polyorganosiloxane compounds X and Y comprise at least 2 trialkoxysilane end groups per polymer molecule, said groups having the following formula:

-ZSiR¹ₓ(OR)₃₋ₓ, (VI)

wherein:

the radicals R independently are chosen from methyl, ethyl, n-propyl, isopropyl, n-butyl, sec-butyl and isobutyl groups, and in one embodiment are chosen from methyl and ethyl groups,

R¹ is chosen from methyl and ethyl groups,

x is equal to 0 or 1, for example x is equal to 0, and

Z is chosen from: divalent hydrocarbon-based groups not comprising any ethylenic unsaturation and comprising from 1 to 18 carbon atoms (alkylene groups), for example from 2 to 18 carbon atoms, and combinations of divalent hydrocarbon-based radicals and of siloxane segments of formula (IX) below:

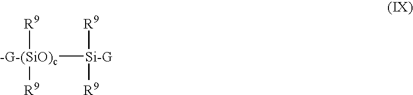

wherein

R⁹ is as described above,

G is a divalent hydrocarbon-based radical not comprising any ethylenic unsaturation and comprising from 1 to 18 carbon atoms, for example from 2 to 18 carbon atoms, and

c is an integer ranging from 1 to 6.

In one embodiment, Z and G may be chosen from alkylene groups such as methylene, ethylene, propylene, butylene, pentylene and hexylene, and arylene groups such as phenylene. In a further embodiment, Z is an alkylene group, for example, ethylene.

These polymers may contain on average at least 1.2 trialkoxysilane end groups or end chains per molecule, for example on average at least 1.5 trialkoxysilane end groups per molecule. Since these polymers may contain at least 1.2 trialkoxysilane end groups per molecule, some may comprise other types of end groups, such as end groups of formula CH₂═CH═SiR⁹₂— or of formula R⁶₃—Si—, wherein R⁹ is as defined above and each group R⁶ is independently chosen from groups R⁹ and vinyl. Examples of such end groups include, but are not limited to, trimethoxysilane, triethoxysilane, vinyidimethoxysilane and vinylmethyloxyphenylsi lane groups.

Further examples of such polymers include those described in U.S. Pat. Nos. 3,175,993, 4,772,675, 4,871,827, 4,888,380, 4,898,910, 4,906,719 and 4,962,174, the contents of which are incorporated herein by reference.

In one embodiment, compound X and/or Y is a polymer of formula

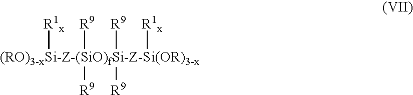

wherein R, R¹, R⁹, Z, x and f are as defined above.

In another embodiment, compounds X and/or Y may also comprise a mixture of polymer of formula (VII) above with polymers of formula (VIII) below:

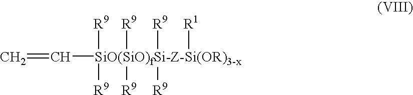

wherein R, R¹, R⁹, Z, x and f are as defined above.

When at least one polyorganosiloxane compound X and/or Y comprising at least one alkoxysilane group comprises such a mixture, the various polyorganosiloxanes are present in amounts such that the organosiylyl end chains represent less than 40% relative to the number of end chains, for example less than 25% relative to the number of the end chains.

In at least one embodiment, the polyorganosiloxane compounds X and/or Y are those of formula (VII) described above. Such compounds X and/or Y are described, for example, in International Application WO 01/96450.

As indicated above, compounds X and Y may be identical or different.

In one embodiment, one of the two reactive compounds X or Y is of silicone nature and the other is of organic nature. For example, compound X may be chosen from organic oligomers or polymers and organic/silicone hybrid oligomers or polymers, wherein said polymers or oligomers comprise at least two alkoxysilane groups, and Y may be chosen from silicone compounds such as the polyorganosiloxanes described above. In a further embodiment, the at least one organic oligomer or polymer is chosen from vinyl, (meth)acrylic, polyester, polyamide, polyurethane and/or polyurea, polyether, polyolefin or perfluoropolyether oligomers or polymers, and hyperbranched organic dendrimers and polymers, and mixtures thereof.

In one embodiment, the at least one organic polymer of vinyl or (meth)acrylic nature comprising at least one alkoxysilane side group may be obtained via copolymerization of at least one organic vinyl or (meth)acrylic monomer with at least one of (meth)acryloxypropyltrimethoxysilanes, vinyltrimethoxysilanes, vinyltriethoxysilanes, allyltrimethoxysilanes, etc.

Examples of such polymers include, but are not limited to, the (meth)acrylic polymers described in the document by Kusabe, M., Pitture e Verniei—European Coating; 12-B, pages 43-49, 2005, such as the polyacrylates comprising alkoxysilane groups referenced as MAX from Kaneka and those described in the publication by Probster, M., Adhesion-Kleben & Dichten, 2004, 481 (1-2), pages 12-14.

The organic polymers resulting from a polycondensation or a polyaddition, such as polyesters, polyamides, polyurethanes and/or polyureas, and polyethers, and bearing at least one alkoxysilane side and/or end group, may result, for example, from the reaction of an oligomeric prepolymer as described above with at least one of the following silane coreagents bearing at least one alkoxysilane group: aminopropyltrimethoxysilane, aminopropyltriethoxysilane, aminoethylaminopropyltrimethoxysilane, glycidoxypropyltrimethoxysilane, glycidoxypropyltriethoxysilane, epoxycyclohexylethyltrimethoxysilane, and/or mercaptopropyltrimethoxysilane.

Examples of polyethers and polyisobutylenes comprising alkoxysilane groups are described in the publication by Kusabe, M., Pitture e Verniei—European Coating; 12-B, pages 43-49, 2005. Examples of polyurethanes comprising alkoxysilane end groups include, but are not limited to those described in the document by Probster, M., Adhesion-Kleben & Dichten, 2004, 481 (1-2) pages 12-14 or those described in the document by Landon, S., Pitture e Verniei vol. 73, No. 11, pages 18-24, 1997 or in the document by Huang, Mowo, Pitture e Verniei vol. 5, 2000, pages 61-67; for example the polyurethanes comprising alkoxysilane groups from OSI-WITCO-GE.

Examples of polyorganosiloxane compounds X and/or Y include, but are not limited to, resins of MQ or MT type themselves comprising at least one alkoxysilane and/or silanol end, for instance the poly(isobutylsilsesquioxane) resins functionalized with silanol groups sold under the reference SST-S7C41 (3 Si—OH groups) by the company Gelest.

2a—Additional Reactive Compound

In one embodiment, one of the compositions disclosed herein may also comprise at least one additional reactive compound comprising at least two alkoxysilane or silanol groups.

Examples of additional reactive compounds include, but are not limited to:

. . . at least one of organic or mineral particles comprising at their surface alkoxysilane and/or silanol groups, for instance fillers surface-treated with such groups.

2b—Catalyst

The condensation reaction may be performed in the presence of at least one metal-based catalyst that may be present in at least one of the compositions comprising X and/or Y or in a separate composition. In one embodiment, at least one metal-based catalyst is a titanium-based catalyst.

Examples of metal-based catalysts include, but are not limited to the tetraalkoxytitanium-based catalysts of formula

Ti(OR²)_{y}(OR³)_{4-y},

wherein R² is chosen from tertiary alkyl radicals such as tert-butyl, tert-amyl and 2,4-dimethyl-3-pentyl; R³ is an alkyl radical comprising from 1 to 6 carbon atoms, such as methyl, ethyl, n-propyl, isopropyl, n-butyl, sec-butyl or hexyl groups and y is a number ranging from 3 to 4, for example from 3.4 to 4.

The catalyst may be present in at least one of the compositions disclosed herein in a content ranging from 0.0001% to 20% by weight relative to the total weight of the at least one composition comprising it.

### 2c —Diluent

Compositions comprising compounds X and/or Y as disclosed herein may also comprise at least one volatile silicone oil (or diluent) for reducing the viscosity of the composition. The at least one oil may be chosen, for instance, from short-chain linear silicones such as hexamethyldisiloxane or octamethyltrisiloxane, and cyclic silicones such as octamethylcyclotetrasiloxane and decamethylcyclopentasiloxane, and mixtures thereof.

This silicone oil may be present in an amount ranging from 5% to 95%, for example from 10% to 80% by weight relative to the weight of each composition.

Examples of compounds X and Y comprising alkoxysilane groups and reacting via condensation include, but are not limited to the mixtures A′ and B′ below sold by the company Dow Corning.

### Mixture A′:

| Ingredient (INCI name) | CAS No. | Content (%) | Function |
|---|---|---|---|
| Bis-Trimethoxysiloxyethyl | PMN87176 | 25-45 | Polymer |
| Tetramethyldisiloxyethyl | | | |
| Dimethicone* | | | |
| Silica Silylate | 68909-20-6 | 5-20 | Filler |
| Disiloxane | 107-46-0 | 30-70 | Solvent |

*corresponds to identical compounds X and Y

### Mixture B′:

| Ingredient (INCI name) | CAS No. | Content (%) | Function |
|---|---|---|---|
| Disiloxane | 107-46-0 | 80-99 | Solvent |
| Tetra T Butyl Titanate | — | 1-20 | Catalyst |

It should moreover be noted that the identical compounds X and Y are combined in the mixture A′.

### 3—Crosslinking in the Presence of Peroxide:

In one embodiment, at least one crosslinking reaction may be performed by heating to a temperature of greater than or equal to 50° C., such as greater than or equal to 80° C., which, in one embodiment, may be up to 120° C. The identical or different compounds X and Y comprise in this case at least two —CH₃ side groups and/or at least two side chains bearing a —CH₃ group.

In at least one embodiment, compounds X and Y are silicone compounds and may be chosen, for example, from high molecular weight non-volatile linear polydimethylsiloxanes, with a degree of polymerization of greater than 6, comprising at least two —CH₃ side groups bonded to the silicon atom and/or at least two side chains bearing at least one —CH₃ group. Examples include, but are not limited to, polymers described in the “Reactive Silicones” catalogue from the company Gelest Inc., Edition 2004, page 6, such as vinylmethylsiloxane-dimethylsiloxane copolymers (also referred to as gums) with molecular weights ranging from 500,000 to 900,000 and viscosities greater than 2,000,000 cSt.

Examples of peroxides that may be used in the context of the present disclosure include, but are not limited to, benzoyl peroxide and 2,4-dichlorobenzoyl peroxide, and mixtures thereof.

According to one embodiment, the hydrosilylation reaction or the condensation reaction, or alternatively the crosslinking reaction in the presence of at least one peroxide, between compounds X and Y is accelerated by supplying heat, for example by raising the temperature of the system to a temperature ranging from 25° C. to 180° C. The system may, in at least one embodiment, react on the skin.

In general, irrespective of the type of reaction via which compounds X and Y react together, the mole percentage of X relative to all of the compounds X and Y, i.e. the ratio X/(X+Y)×100, may range from 5% to 95%, for example from 10% to 90% or from 20% to 80%.

Similarly, the mole percentage of Y relative to all of the compounds X and Y, i.e. the ratio Y/(X+Y)×100, may range from 5% to 95%, for example from 10% to 90% or from 20% to 80%.

Compound X may have a weight-average molecular mass (Mw) ranging from 150 to 1,000,000, for example from 200 to 800,000 or from 200 to 250,000.

Compound Y may have a weight-average molecular mass (Mw) ranging from 200 to 1,000,000, for example from 300 to 800,000 or from 500 to 250,000.

Compound X may be present in an amount ranging from 0.5% to 95% by weight, for example, from 1% to 90% or from 5% to 80%, relative to the total weight of the useful compositions or relative to the total weight of the composition when X and Y are present in the same composition.

Compound Y may be present in an amount ranging from 0.05% to 95% by weight, for example, from 0.1% to 90% or from 0.2% to 80%, relative to the total weight of the useful compositions or relative to the total weight of the composition when X and Y are present in the same composition.

The ratio between the compounds X and Y may be varied so as to modify the rate of reaction and thus the rate of formation of the film, or alternatively so as to adapt the properties of the film formed (for example its adhesive properties) according to the desired application.

In at least one embodiment, compounds X and Y may be present in a mole ratio X/Y ranging from 0.05 to 20, for example from 0.1 to 10.

### Texturizing Agents

In one embodiment, the composition applied to the hair fibers may also comprise at least one texturizing agent (filler), other than the pigments, fluorescent dyes or optical brighteners present in the composition.

As used herein, the term “texturizing agents” means mineral or synthetic, lamellar or non-lamellar, water-insoluble particles.

By way of example, and not by way of limitation, these texturizing agents may be colloidal calcium carbonate, which may or may not be treated with stearic acid or stearate, silica such as fumed silicas, precipitated silicas and silicas treated to make them hydrophobic, ground quartz, alumina, aluminium hydroxide, titanium dioxide, diatomaceous earth, iron oxide or carbon black.

Further non-limiting examples of texturizing agents include talc, mica, kaolin, polyamide (Nylon®) powders (Orgasol from Atochem), polyethylene powders, tetrafluoroethylene polymer (Teflon®) powders, starch, polymer microspheres such as those of polyvinylidene chloride/acrylonitrile, for instance Expancel (Nobel Industrie) and acrylic acid copolymers (Polytrap® from the company Dow Corning).

In at least one embodiment, at least one texturizing agent is chosen from synthetic silicas whose surface is modified with at least one silicone compound to make the surface hydrophobic. These fillers may be distinguished from each other by their surface properties, the silicone compounds used to treat the silica, and the way wherein the surface treatment is performed.

Such agents may make it possible to modify the viscosity of the formulation obtained with compounds X and/or Y and/or to modify the properties of the material obtained.

In at least one embodiment, the texturizing agent is chosen from silica, calcium carbonate and resin-based agents.

Examples of useful texturizing agents include, but are not limited to, the treated silicas Cab-O—Sil®TS-530, Aerosil®R8200 and Wacker HDX H2000.

The texturizing agents may be present in an amount ranging from 0 to 48% by weight relative to the total weight of the composition, for example from 0.01% to 30% by weight or from 0.02% to 20% by weight relative to the total weight of the composition.

### II) Fluorescent Compounds: Fluorescent Dyes and Optional Optical Brighteners:

The compositions disclosed herein comprise at least one fluorescent dye in soluble form or in pigment form. These compositions may also comprise at least one optical brightener in soluble form or in pigment form.

As used herein, the term “fluorescent compound” means fluorescent dyes and, when they are present, optical brighteners.

As used herein, the term “pigment” means any fluorescent compound that has in the medium a solubility in water of less than 0.01% at 20° C. and at atmospheric pressure (760 mmHg).

As used herein, the term “soluble fluorescent compound” means a fluorescent compound that has a solubility in the medium of greater than 0.01% at 20° C., for example greater than 0.05%, at atmospheric pressure.

As used herein, “fluorescent dyes” are fluorescent compounds that absorb in the visible radiation range generally between 400 and 800 nm and that are capable of re-emitting light in the visible range at a higher wavelength. By definition, these dyes are colored species since they absorb visible light.

According to one embodiment, the fluorescent dyes that are useful in the context of the present disclosure re-emit orange fluorescent light. In a further embodiment, the fluorescent dyes may have a maximum reemission wavelength ranging from 500 to 700 nm.

Examples of fluorescent dyes include, but are not limited to, the compounds known in the art described, for example, in the following publication: Ullmann's Encyclopedia of Industrial Chemistry Release 2004, 7th edition, chapter “Fluorescent Dyes”.

Other examples of fluorescent compounds that may be used in the context of the present disclosure are compounds known in the art. They may be described, for example, in French Patent No. 2 830 189.

Examples of soluble fluorescent compounds include, but are not limited to, those belonging to the following families: naphthalimides, coumarins, and xanthenes such as xanthenodiquinolizines and azaxanthenes; naphtholactams; azlactones; oxazines; thiazines; dioxazines; azo compounds; azomethines; methines; pyrazines; stilbenes; ketopyrroles; and pyrenes.

Examples of fluorescent dyes include, but are not limited to:

the methine derivatives of formula:

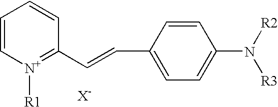

wherein R₁, R₂ and R₃, independently of each other, are chosen from hydrogen atoms; halogen atoms; C₆-C₃₀ aryl groups; hydroxyl groups; cyano groups; nitro groups; sulfo groups; amino groups; acylamino groups; di(C₁-C₆)alkylamino groups; dihydroxy(C₁-C₆)alkylamino groups; (C₁-C₆)alkylhydroxy(C₁-C₆)alkylamino groups; (C₁-C₆)alkoxy groups; (C₁-C₆)alkoxycarbonyl groups; carboxy(C₁-C₆)alkoxy groups; piperidinosulfonyl groups; pyrrolidino groups; (C₁-C₆)alkylhalo(C₁-C₆)alkylamino groups; benzoyl(C₁-C₆)alkyl groups; vinyl groups; formyl groups; C₆-C₃₀ aryl radicals optionally substituted with at least one group chosen from hydroxyl groups, linear, branched or cyclic C₁-C₆ alkoxy groups, linear, branched or cyclic alkyl groups comprising from 1 to 22 carbon atoms, optionally substituted with at least one hydroxyl, amino or C₁-C₆ alkoxy group; linear, branched or cyclic alkyl radicals comprising from 1 to 22 carbon atoms, for example from 1 to 6 carbon atoms, optionally substituted with at least one group chosen from hydroxyl groups, amino groups, linear, branched or cyclic C₁-C₆ alkoxy groups, optionally substituted aryl groups, carboxyl groups, sulfo groups and halogen atoms, wherein at least one alkyl radical is optionally interrupted with at least one heteroatom, such as nitrogen, sulfur and oxygen; two of the substituents R₂ and R₃ may form, with the carbon atoms to which they are attached, a C₆-C₃₀ aromatic or heterocyclic nucleus comprising in total from 5 to 30 ring members and from 1 to 5 heteroatoms; wherein the ring is fused or non-fused, and wherein the ring and/or the possible fused ring are unsubstituted or substituted with at least one group chosen from C₁-C₄ alkyl, (C₁-C₄)alkoxy(C₁-C₄)alkyl, amino, di(C₁-C₄)alkylamino, halogen, phenyl, carboxyl and tri(C₁-C₄)alkylammonio(C₁-C₄)alkyl groups, and
X⁻ is an organic or mineral anion. Examples of X⁻ include, but are not limited to, chloride, bromide, iodide, methosulfate, ethosulfate, mesylate, tosylate and acetate ions, and the salts of simple organic acids such as lactate, oleate, benzoate, perchlorate and triflate.

In one embodiment, the at least one fluorescent dye is chosen from 2-[2-(4-dimethylamino)phenylethenyl]-1-ethylpyridinium salts:

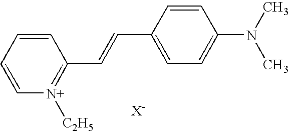

wherein X⁻ is an anion that may be chosen from halides, alkyl sulfates such as methyl sulfate and ethyl sulfate; alkoxysulfates such as methoxysulfate and ethoxysulfate; phosphates; lactate; citrate; acetate; and tartrate.

In one embodiment, the counterion may be chosen from chloride and ethosulfate.

In another embodiment, the at least one fluorescent dye is chosen from compounds having the formulae below:

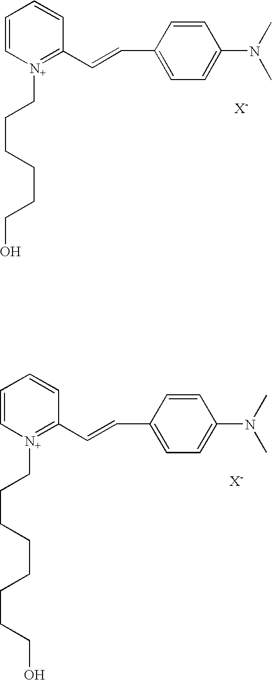

wherein X⁻ has the same meanings as defined above;

Brilliant Yellow B6GL sold by the company Sandoz and having the following structure:

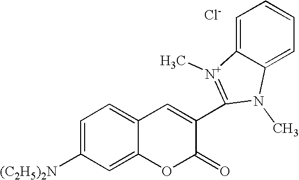

the methine hemicyanin below:

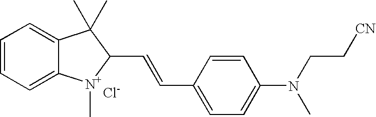

Basic Yellow 2, or Auramine O, sold by the companies Prolabo, Aldrich or Carlo Erba and having the following structure:

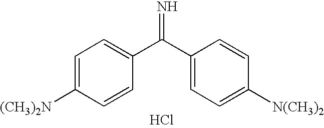

and 4,4′-(imidocarbonyl)bis(N,N-dimethylaniline) monohydrochloride

In yet another embodiment, the at least one fluorescent dye is chosen from compounds having the following formula:

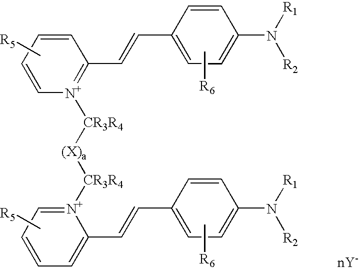

wherein:

R₁ and R₂, which may be identical or different, are chosen from:
hydrogen atoms;
linear or branched alkyl radicals comprising from 1 to 10 carbon atoms, for example from 1 to 4 carbon atoms, optionally interrupted and/or substituted with at least one heteroatom and/or group comprising at least one heteroatom and/or substituted with at least one halogen atom; and
aryl or arylalkyl radicals, wherein the aryl group comprises 6 carbon atoms and the alkyl radicals comprise from 1 to 4 carbon atoms; the aryl radicals optionally being substituted with at least one linear or branched alkyl radical comprising from 1 to 4 carbon atoms optionally interrupted and/or substituted with at least one heteroatom and/or group comprising at least one heteroatom and/or substituted with at least one halogen atom; and
R₁ and R₂ may optionally be linked so as to form a heterocycle with the nitrogen atom and may comprise at least one other heteroatom, the heterocycle optionally being substituted with at least one linear or branched alkyl radical, for example comprising from 1 to 4 carbon atoms and optionally being interrupted and/or substituted with at least one heteroatom and/or group comprising at least one heteroatom and/or substituted with at least one halogen atom;
R₁ or R₂ may optionally be engaged in a heterocycle comprising at least one nitrogen atom and one of the carbon atoms of the phenyl group comprising said at least one nitrogen atom;

R₃ and R₄, which may be identical or different, are chosen from hydrogen atoms and alkyl radicals comprising from 1 to 4 carbon atoms;

R₅, which may be identical or different, is chosen from hydrogen atoms, halogen atoms and linear or branched alkyl radicals comprising from 1 to 4 carbon atoms, optionally interrupted with at least one heteroatom;

R₆, which may be identical or different, is chosen from hydrogen atoms; halogen atoms; and linear or branched alkyl radicals comprising from 1 to 4 carbon atoms, optionally substituted and/or interrupted with at least one heteroatom and/or group comprising at least one heteroatom and/or substituted with at least one halogen atom;

X is chosen from:
linear or branched alkyl radicals comprising from 1 to 14 carbon atoms or at least one alkenyl radical comprising from 2 to 14 carbon atoms, optionally interrupted and/or substituted with at least one heteroatom and/or group comprising at least one heteroatom and/or substituted with at least one halogen atom;
5- or 6-membered heterocyclic radicals optionally substituted with at least one linear or branched alkyl radical comprising from 1 to 14 carbon atoms, optionally substituted with at least one heteroatom; with at least one linear or branched aminoalkyl radical comprising from 1 to 4 carbon atoms, optionally substituted with at least one heteroatom; and/or with at least one halogen atom;
fused or non-fused aromatic or diaromatic radicals, optionally separated with at least one alkyl radical comprising from 1 to 4 carbon atoms, the at least one aryl radical optionally being substituted with at least one halogen atom or with at least one alkyl radical comprising from 1 to 10 carbon atoms optionally substituted and/or interrupted with at least one heteroatom and/or group comprising at least one heteroatom;
dicarbonyl radicals;
the groups X possibly comprising at least one cationic charge;

a is equal to 0 or 1;

Y⁻, which may be identical or different, represents an organic or mineral anion; and

n is an integer at least equal to 2 and at most equal to the number of cationic charges present in the fluorescent compound.

As used herein, the term “heteroatom” means an oxygen or nitrogen atom.

Examples of groups bearing heteroatoms include, but are not limited to hydroxyl, alkoxy, carbonyl, amino, ammonium, amido (—N—CO—) and carboxyl (—O—CO— or —CO—O—) groups.

In one embodiment, the alkenyl groups comprise at least one unsaturated carbon-carbon bond (—C═C—), such as only one carbon-carbon double bond.

In one embodiment, in the latter general formula, the radicals R₁ and R₂, which may be identical or different, are chosen from:

hydrogen atoms;

alkyl radicals comprising from 1 to 10 carbon atoms, for example from 1 to 6 carbon atoms or from 1 to 4 carbon atoms, optionally interrupted with an oxygen atom or optionally substituted with at least one hydroxyl, amino or ammonium radical or a chlorine or fluorine atom;

benzyl or phenyl radicals optionally substituted with an alkyl or alkoxy radical comprising from 1 to 4 carbon atoms, for example 1 or 2 carbon atoms;

with the nitrogen atom, heterocyclic radicals of the pyrrolo, pyrrolidino, imidazolino, imidazolo, imidazolium, pyrazolino, piperazino, morpholino, morpholo, pyrazolo or triazolo types, optionally substituted with at least one linear or branched alkyl radical comprising from 1 to 4 carbon atoms optionally interrupted and/or substituted with at least one nitrogen and/or oxygen atom and/or group bearing at least one nitrogen and/or oxygen atom.

As regards the abovementioned amino or ammonium radicals, the radicals borne by the nitrogen atom may be identical or different and may, in one embodiment, be chosen from hydrogen atoms, C₁-C₁₀, for example C₁-C₄, alkyl radicals and arylalkyl radicals wherein, in a further embodiment, at least one aryl radical comprises 6 carbon atoms and at least one alkyl radical comprises from 1 to 10 carbon atoms, for example from 1 to 4 carbon atoms.

According to one embodiment of the present disclosure, the radicals R₁ and R₂, which may be identical or different, are chosen from hydrogen atoms; linear or branched C₁-C₆ alkyl radicals; C₂-C₆ alkyl radicals substituted with at least one hydroxyl radical; C₂-C₆ alkyl radicals comprising at least one amino or ammonium group; C₂-C₆ chloroalkyl radicals; C₂-C₆ alkyl radicals interrupted with at least one oxygen atom or at least one group comprising an oxygen atom (for example ester); aromatic radicals, for instance phenyl, benzyl or 4-methylphenyl; heterocyclic radicals such as pyrrolo, pyrrolidino, imidazolo, imidazolino, imidazolium, piperazino, morpholo, morpholino, pyrazolo or triazolo radicals, optionally substituted with at least one from C₁-C₆ alkyl or aromatic radical.

In at least one embodiment, the radicals R₁ and R₂, which may be identical or different, are chosen from hydrogen atoms, linear or branched C₁-C₆ alkyl radicals such as methyl, ethyl, n-butyl or n-propyl radicals; 2-hydroxyethyl; alkyltrimethylammonium or alkyltriethylammonium radicals, wherein the alkyl radical is a linear C₂-C₆ alkyl radical; (di)alkylmethylamino or (di)alkylethylamino radicals, wherein the alkyl radical is a linear C₂-C₆ alkyl radical; —CH₂CH₂Cl; —(CH₂)ₙ—OCH₃ or —(CH₂)ₙ—OCH₂CH₃ wherein n is an integer ranging from 2 to 6; —CH₂CH₂—OCOCH₃; and —CH₂CH₂COOCH₃.

In another embodiment, the radicals R₁ and R₂, which may be identical or different, and are in one embodiment identical, are chosen from methyl radicals and ethyl radicals.

In yet another embodiment, the radicals R₁ and R₂, which may be identical or different, may also represent heterocyclic radicals of the pyrrolidino, 3-aminopyrrolidino, 3-(dimethyl)aminopyrrolidino, 3-(trimethyl)aminopyrrolidino, 2,5-dimethylpyrrolo, 1H-imidazolo, 4-methylpiperazino, 4-benzylpiperazino, morpholo, 3,5-(tert-butyl)-1H-pyrazolo, 1H-pyrazolo or 1H-1,2,4-triazolo type.

In another embodiment, the radicals R₁ and R₂, which may be identical or different, may also represent and be linked so as to form a heterocycle of formulae (I) and (II) below:

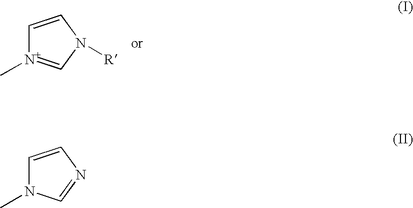

wherein R′ is chosen from hydrogen atoms, C₁-C₃ alkyl radicals, —CH₂CH₂OH, and —CH₂CH₂OCH₃.

In accordance with another embodiment, R₅, which may be identical or different, is chosen from hydrogen atoms, fluorine or chlorine atoms and linear or branched alkyl radicals comprising from 1 to 4 carbon atoms optionally interrupted with at least one oxygen or nitrogen atom.

In one embodiment, wherein the substituent R₅ is other than hydrogen, R₅ may be in position(s) 3 and/or 5 relative to the carbon of the ring bearing the nitrogen substituted with the radicals R₁ and R₂, for example in position 3 relative to that carbon.

In one embodiment, the radicals R₅, which may be identical or different, are chosen from hydrogen atoms; linear or branched C₁-C₄ alkyl radicals; —O—R₅₁ wherein R₅₁ represents a linear C₁-C₄ alkyl radical; —R₅₂—O—CH₃ wherein R₅₂ represents a linear C₂-C₃ alkyl radical; —R₅₃—N(R₅₄)₂ wherein R₅₃ represents a linear C₂-C₃ alkyl radical and R₅₄, which may be identical or different, represent a hydrogen atom or a methyl radical.

In another embodiment, R₅, which may be identical or different, is chosen from hydrogen atoms, and methyl or methoxy groups, for example, R₅ may represent a hydrogen atom.

According to one embodiment, the radicals R₆, which may be identical or different, are chosen from hydrogen atoms; linear or branched C₁-C₄ alkyl radicals; —X wherein X is chosen from chlorine, bromine and fluorine atoms; —R₆₁—O—R₆₂ wherein R₆₁ represents a linear C₂-C₃ alkyl radical and R₆₂ represents a methyl radical; —R₆₃—N(R₆₄)₂ wherein R₆₃ represents a linear C₂-C₃ alkyl radical and R₆₄, which may be identical or different, is chosen from hydrogen atoms and methyl radicals; —N(R₆₅)₂ wherein R₆₅, which may be identical or different, is chosen from hydrogen atoms and linear C₂-C₃ alkyl radicals; —NHCOR₆₆ wherein R₆₆ is chosen from C₁-C₂ alkyl radicals, C₁-C₂ chloroalkyl radicals, and radicals —R₆₇—NH₂ or —R₆₇—NH(CH₃) or —R₆₇—N(CH₃)₂ or —R₆₇—N⁺(CH₃)₃ or —R₆₇—N⁺(CH₂CH₃)₃ wherein R₆₇ represents a C₁-C₂ alkyl radical.

In one embodiment, the substituent R₆, if it is other than hydrogen, may be in position 2 and/or 4 relative to the nitrogen atom of the pyridinium ring, for example in position 4 relative to that nitrogen atom.

In another embodiment, the substituent R₆, which may be identical or different, is chosen from hydrogen atoms and methyl or ethyl radicals. In a further embodiment, R₆ is a hydrogen atom.

In one embodiment, the radicals R₃ and R₄, which may be identical or different, are chosen from hydrogen atoms and alkyl radicals comprising from 1 to 4 carbon atoms, for example methyl radicals. In a further embodiment, R₃ and R₄ each represent a hydrogen atom.

In one embodiment, X, as mentioned above, is chosen from:

linear or branched alkyl radicals comprising from 1 to 14 carbon atoms or alkenyl radicals comprising from 2 to 14 carbon atoms, optionally interrupted and/or substituted with at least one heteroatom, with at least one group bearing at least one heteroatom and/or with at least one halogen atom;
5- or 6-membered heterocyclic radicals optionally substituted with at least one linear or branched alkyl radical comprising from 1 to 14 carbon atoms, with at least one linear or branched aminoalkyl radical comprising from 1 to 4 carbon atoms, optionally substituted with at least one heteroatom; or with at least one halogen atom;

fused or non-fused aromatic or diaromatic radicals, optionally separated with at least one alkyl radical comprising from 1 to 4 carbon atoms, the at least one aryl radical optionally being substituted with at least one halogen atom or with at least one alkyl radical comprising from 1 to 10 carbon atoms optionally substituted and/or interrupted with at least one heteroatom and/or group comprising at least one heteroatom; and

dicarbonyl radicals.

In another embodiment, the group X may additionally bear at least one cationic charge.

In at least one embodiment, the group X may be chosen from linear or branched alkyl radicals comprising from 1 to 14 carbon atoms or alkenyl radicals comprising from 2 to 14 carbon atoms, and may be substituted and/or interrupted with at least one oxygen and/or nitrogen atom, and/or with at least one group bearing at least one heteroatom, and/or with a fluorine or chlorine atom.

In a further embodiment, the group X is chosen from hydroxyl, alkoxy (for example with a radical R of the C₁-C₄ alkyl type), amino, ammonium, amido, carbonyl and carboxyl groups (—COO— or —O—CO—), and in another embodiment X is a radical of alkyloxy type.

In one embodiment, the nitrogen atom, if it is present, may be in a quaternized or non-quaternized form. In this case, the other radical or the other two radicals borne by the quaternized or non-quaternized nitrogen atom may be identical or different and may be chosen from hydrogen atoms and from C₁-C₄ alkyl radicals, for example methyl.

According to another embodiment, the group X represents a 5- to 6-membered heterocyclic radical of the imidazolo, pyrazolo, triazino or pyridino type, optionally substituted with at least one linear or branched alkyl radical comprising from 1 to 14 carbon atoms, for example from 1 to 10 carbon atoms or from 1 to 4 carbon atoms; further comprising at least one linear or branched aminoalkyl radical comprising from 1 to 10 carbon atoms, for example from 1 to 4 carbon atoms, optionally substituted with a group comprising at least one heteroatom (for example a hydroxyl radical), or with at least one halogen atom. In another embodiment, the amino group is linked to the heterocycle.

In accordance with another embodiment, the group X represents an aromatic radical (for example comprising 6 carbon atoms) or a fused or non-fused diaromatic radical (for example comprising from 10 to 12 carbon atoms), possibly separated with at least one alkyl radical comprising from 1 to 4 carbon atoms, the at least one aryl radical optionally being substituted with at least one halogen atom and/or with at least one alkyl radical comprising from 1 to 10 carbon atoms, for example from 1 to 4 carbon atoms, optionally interrupted with at least one oxygen and/or nitrogen atom and/or at least one group comprising at least one heteroatom (for instance a carbonyl, carboxyl, amido, amino or ammonium radical).

In one embodiment, the aromatic radical, for example a phenyl radical, may be linked to the groups CR₃R₄ via bonds in positions 1,2; 1,3 or 1,4, for example in positions 1,3 and 1,4. In another embodiment, if the phenyl radical linked via bonds in positions 1,4 bears one or two substituents, this or these substituent(s) may be located in position 1,4 relative to one of the groups CR₃R₄. In another embodiment, if the phenyl radical linked via bonds in positions 1,3 bears one or two substituents, this or these substituents may be located in positions 1 and/or 3 relative to one of the groups CR₃R₄.

In one embodiment, in the case where the radical is diaromatic, it may be non-fused and may comprise two phenyl radicals possibly separated with a single bond (i.e. a carbon of each of the two rings) or with an alkyl radical, for example of CH₂ or C(CH₃)₂ type. In at least one embodiment, the aromatic radicals do not bear a substituent.

In one embodiment, the diaromatic radical is linked to the groups CR₃R₄ via bonds in positions 4,4′.

Examples of groups X that may be used include, but are not limited to, linear or branched alkyl radicals comprising from 1 to 13 carbon atoms, such as methylene, ethylene, propylene, isopropylene, n-butylene, pentylene and hexylene; 2-hydroxypropylene and 2-hydroxy-n-butylene; from C₁-C₁₃ alkylene radicals substituted or interrupted with at least one nitrogen and/or oxygen atom, and/or groups bearing at least one heteroatom (hydroxyl, amino, ammonium, carbonyl or carboxyl, for example), such as —CH₂CH₂OCH₂CH₂—, 1,6-dideoxy-d-mannitol, —CH₂N⁺(CH₃)₂CH₂—, —CH₂CH₂N⁺(CH₃)₂—(CH₂)₆N⁺(CH₃)₂—CH₂CH₂—, CO—CO—, 3,3-dimethylpentylene, 2-acetoxyethylene, butylene-1,2,3,4-tetraol; —CH═CH—; aromatic or diaromatic radicals substituted with at least one alkyl radical, with at least one group bearing at least one heteroatom and/or with at least one halogen atom, such as 1,4-phenylene, 1,3-phenylene, 1,2-phenylene, 2,6-fluorobenzene, 4,4′-biphenylene, 1,3-(5-methylbenzene), 1,2-bis(2-methoxy)benzene, bis(4-phenyl)methane, methyl 3,4-benzoate and 1,4-bis(amidomethyl)phenyl; radicals of heterocyclic type such as pyridine, or derivatives such as 2,6-bispyridine, imidazole, imidazolium or triazine.

According to another embodiment of the disclosure, the group X is chosen from linear or branched C₁-C₁₃ alkyl radicals; —CH₂CH(OH)CH₂—; —CH₂CH(Cl)CH₂—; —CH₂CH₂—OCOCH₂—; —CH₂CH₂COOCH₂—; —Ra—O—Rb— wherein Ra represents a linear C₂-C₆ alkyl radical and Rb represents a linear C₁-C₂ alkyl radical; —Rc—N(Rd)—Re— wherein Rc represents a C₂-C₆ alkyl radical, Rd represents a hydrogen atom or a C₁-C₂ alkyl radical and Re represents a C₁-C₆ alkyl radical; —Rf—N⁺(Rg)₂-Rh — wherein Rf represents a linear C₂-C₈ alkyl radical, Rg, which may be identical, represents a C₁-C₂ alkyl radical and Rh represents a linear C₁-C₆ alkyl radical; and —CO—CO—.

In another embodiment the group X may represent an imidazole radical, optionally substituted with at least one alkyl radical comprising from 1 to 14 carbon atoms, for example from 1 to 10 carbon atoms or from 1 to 4 carbon atoms, for example the divalent radicals having the following formula:

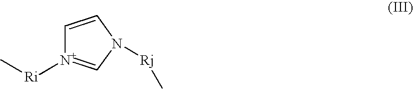

wherein Ri and Rj, which may be identical or different, represent a linear C₁-C₆ alkyl radical;

In another embodiment, the group X may be chosen from the divalent triazine-based radicals below:

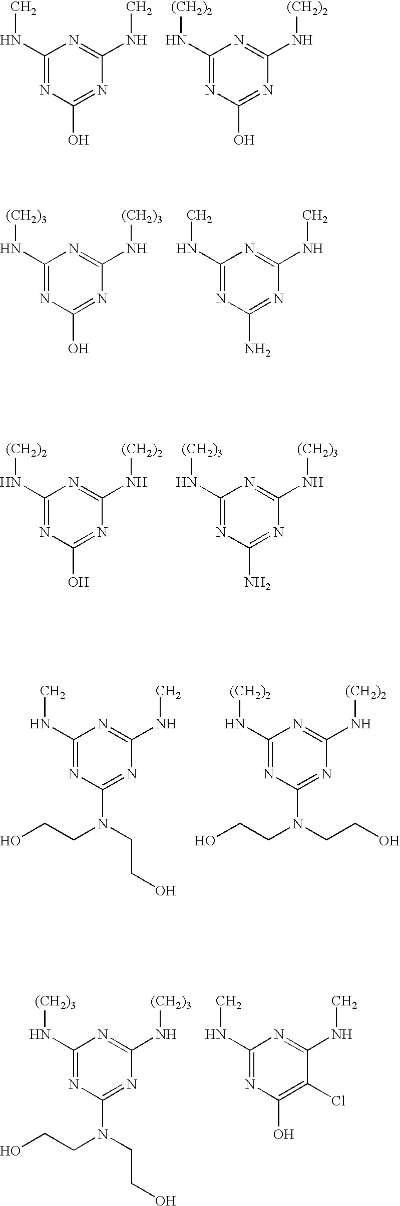

According to another embodiment, the group X may be chosen from the divalent aromatic radicals below:

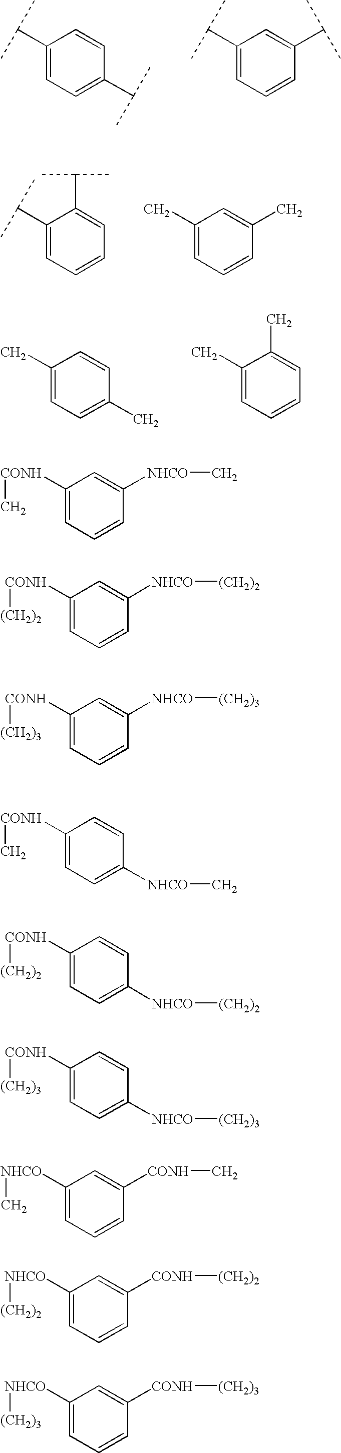

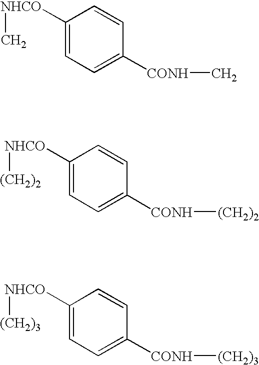

In the general formula of these fluorescent compounds, Y represents an organic or mineral anion. If there are several anions Y⁻, these anions may be identical or different.

Examples of anions of mineral origin include, but are not limited to, anions derived from halogen atoms, such as chlorides, or iodides, sulfates or bisulfates, nitrates, phosphates, hydrogen phosphates, dihydrogen phosphates, carbonates and bicarbonates.

Examples of anions of organic origin include, but are not limited to, anions derived from the salts of saturated or unsaturated, aromatic or non-aromatic monocarboxylic or polycarboxylic, sulfonic or sulfuric acids, optionally substituted with at least one hydroxyl or amino radical, or halogen atoms. Non-limiting examples that are suitable for use herein include acetates, hydroxyacetates, aminoacetates, (tri)chloro-acetates, benzoxyacetates, propionates and derivatives bearing a chlorine atom, fumarates, oxalates, acrylates, malonates, succinates, lactates, tartrates, glycolates, citrates, benzoates and derivatives bearing a methyl or amino radical, alkyl sulfates, tosylates, benzenesulfonates, toluenesulfonates, etc.

In one embodiment, the anion Y, which may be identical or different, is chosen from chloride, sulfate, methosulfate and ethosulfate groups.

In one embodiment, the integer n is at least equal to 2 and at most equal to the number of cationic charges present in the fluorescent compound.

In another embodiment, the fluorescent compounds that have just been described in detail are symmetrical compounds.

In one embodiment, these compounds may be synthesized by reacting, in a first step, α-picoline with a reagent comprising two leaving groups that may be chosen from halogen atoms, for example bromine, or optionally chlorine, and groups of tolylsulfonyl or methanesulfonyl type.

This first step may optionally take place in the presence of a solvent, for instance dimethylformamide.

The number of moles of α-picoline is in about 2 per mole of reagent comprising the leaving groups.

In addition, the reaction is usually performed at the reflux temperature of the reagent and/or of the solvent if a solvent is present.

The product derived from this first step may then be placed in contact with a corresponding aldehyde having the following formula:

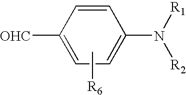

wherein R₁, R₂ and R₆ have the same meanings as indicated above.

In this case also, the reaction may be performed in the presence of a suitable solvent, which may be at reflux.

In one embodiment, the radicals R₁ and R₂ of the aldehyde may have the meanings indicated in the general formula detailed above.

In another embodiment, it is also possible to use an aldehyde wherein the radicals R₁ and R₂ represent hydrogen atoms and to perform, in accordance with standard methods, the substitution of these hydrogen atoms with suitable radicals as described in the definition of the general formula above, once the second step is complete.

Examples of such syntheses include, but are not limited to, those described in U.S. Pat. No. 4,256,458.

Non-limiting mention may also be made of the following compounds:

thioxanthene derivatives such as:

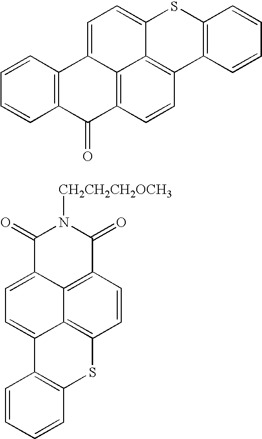

Samaron, Brilliant yellow H6GL, C.I. 56235 Disperse yellow 105;

rhodamines such as:

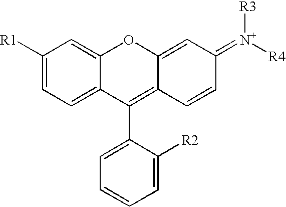

wherein R1, R2, R3 and R4 are as defined above, for example:

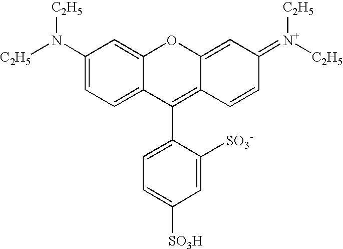

### Sulforhodamine B C.I. 45100 Acid Red 52;

stilbazolium dimers of formulae:

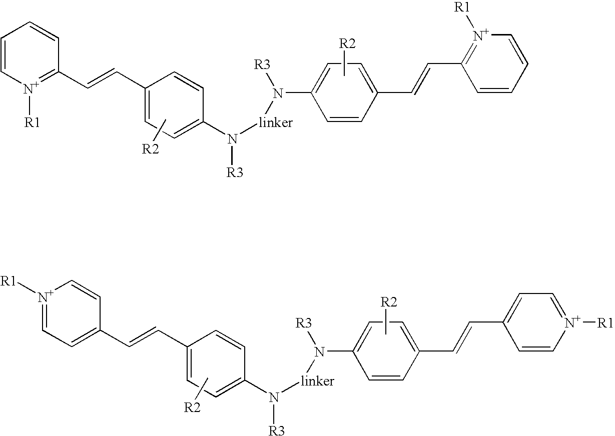

wherein:

R1, R2 and R3, independently of each other, are chosen from hydrogen atoms; halogen atoms; C6-C30 aryl groups; hydroxyl groups; cyano groups; nitro groups; sulfo groups; amino groups; acylamino groups; di(C1-C6)alkylamino groups; dihydroxy(C1-C6) alkylamino groups; (C1-C6)alkylhydroxy(C1-C6)alkylamino groups; (C1-C6)alkoxy groups; (C1-C6)alkoxycarbonyl groups; carboxy(C1-C6)alkoxy groups; piperidinosulfonyl groups; pyrrolidino groups; (C1-C6)alkylhalo(C1-C6)alkylamino groups; benzoyl(C1-C6)alkyl groups; vinyl groups; formyl groups; C6-C30 aryl radicals optionally substituted with at least one group chosen from hydroxyl groups, linear, branched or cyclic C1-C6 alkoxy groups, and linear, branched or cyclic alkyl groups comprising from 1 to 22 carbon atoms, optionally substituted with at least one hydroxyl, amino or C1-C6 alkoxy group; and linear, branched or cyclic alkyl radicals comprising from 1 to 22 carbon atoms, for example from 1 to 6 carbon atoms, optionally substituted with at least one group chosen from hydroxyl groups, amino groups, linear, branched or cyclic from C1-C6 alkoxy groups, optionally substituted aryl groups, carboxyl groups, sulfo groups or halogen atoms, the alkyl radical possibly, being interrupted with at least one heteroatom, such as nitrogen, sulfur or oxygen;

“Linker” represents a saturated or unsaturated, aliphatic or alicyclic C1-C12 hydrocarbon-based chain, at least one carbon atom of the hydrocarbon-based chain possibly being replaced with at least one oxygen atom, at least one group NR wherein R is chosen from hydrogen atoms or alkyl radicals, the hydrocarbon-based chain not comprising any diazo, nitro, nitroso or peroxide groups, and the hydrocarbon-based chain not ending at either of its ends with a heteroatom; and

X⁻ represents an anionic counterion as defined above;
the methine trimers and tetramers below

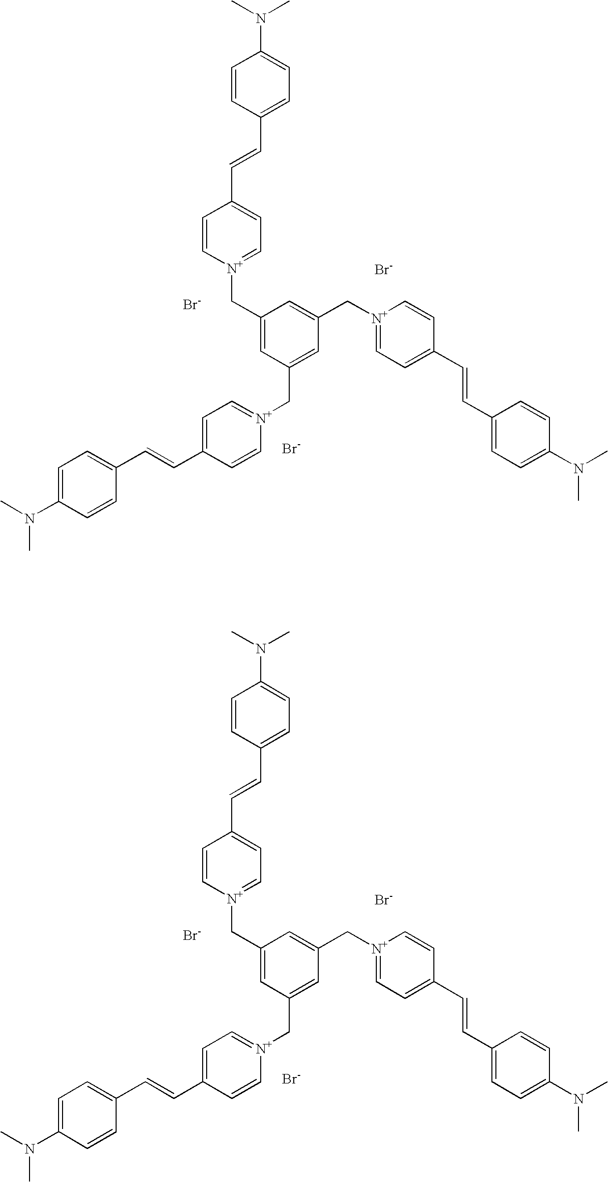

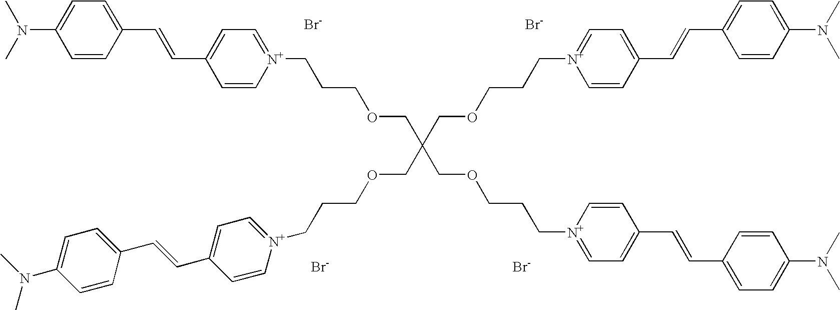

xanthene derivatives such as:

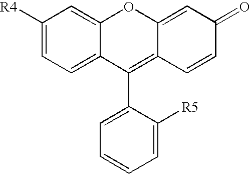

wherein R4 and R5, independently of each other, are chosen from hydrogen atoms; halogen atoms; C6-C30 aryl groups; hydroxyl groups; cyano groups; nitro groups; sulfo groups; amino groups; acylamino groups; di(C1-C6)alkylamino groups; dihydroxy(C1-C6) alkylamino groups; (C1-C6)alkylhydroxy(C1-C6)alkylamino groups; (C1-C6)alkoxy groups; (C1-C6)alkoxycarbonyl groups; carboxy(C1-C6)alkoxy groups; piperidinosulfonyl groups; pyrrolidino groups; (C1-C6)alkylhalo(C1-C6)alkylamino groups; benzoyl(C1-C6)alkyl groups; vinyl groups; formyl groups; C6-C30 aryl radicals optionally substituted with at least one group chosen from hydroxyl groups, linear, branched or cyclic C1-C6 alkoxy groups, and linear, branched or cyclic alkyl groups comprising from 1 to 22 carbon atoms, optionally substituted with at least one hydroxyl, amino or from C1-C6 alkoxy group; and linear, branched or cyclic alkyl radicals comprising from 1 to 22 carbon atoms, for example from 1 to 6 carbon atoms, optionally substituted with at least one group chosen from hydroxyl groups, amino groups, linear, branched or cyclic C1-C6 alkoxy groups, optionally substituted aryl groups, carboxyl groups, sulfo groups and halogen atoms, the alkyl radical possibly being interrupted with at least one heteroatom, such as nitrogen, sulfur or oxygen.

In at least one embodiment, the composition of the disclosure may also comprise at least one optical brightener. Optical brighteners that are useful in the present disclosure, also known as “brighteners”, “fluorescent brighteners”, “fluorescent brightening agents”, “FWA” or “fluorescent whitening agents”, “whiteners” or “fluorescent whiteners”, are colorless compounds since they do not absorb visible light, but only ultraviolet light (wavelengths ranging from 200 to 400 nanometers), and convert the absorbed energy into fluorescent light of longer wavelength emitted in the visible part of the spectrum; in general in the blue and/or green range, i.e. at wavelengths ranging from 400 to 550 nanometers.

Examples of optical brighteners are known to those skilled in the art. They are described, for example, in Ullmann's Encyclopedia of Industrial Chemistry (2002) “Optical Brighteners” and Kirk-Othmer Encyclopedia of Chemical Technology (1995); “Fluorescent Whitening Agents”.

In one embodiment, the optical brightener that may be present in the composition used in the disclosure is chosen from compounds that absorb light in the ultraviolet part of the spectrum, for example in the UVA region, at a wavelength of between 300 and 390 nm. In a further embodiment, these compounds re-emit a fluorescent light in the visible spectrum, of between 400 and 525 nm.

Examples of optical brighteners that may be used include, but are not limited to, stilbene derivatives, coumarin derivatives, oxazole and benzoxazole derivatives, and imidazole derivatives.

Further examples of optical brighteners include, but are not limited to:

the stilbene derivative of naphthotriazole (Tinopal GS from Ciba), disodium 4,4′-distyrylbiphenyl sulfonate (CTFA name: disodium distyrylbiphenyl disulfonate; Tinopal CBS-X from Ciba: sodium 4,4′-bis[(4,6-dianilino-1,3,5-triazin-2-yl)amino]stilbene-2,2′-disulfonate), the cationic derivative of aminocoumarin (Tinopal SWN Conc. from Ciba), diethylaminomethylcoumarin, 4-methyl-7-diethylcoumarin, sodium 4,4′-bis[(4,6-dianilino-1,3,5-triazin-2-yl)amino]stilbene-2,2′-disulfonate (Tinopal SOP from Ciba), 4,4′-bis[(4-anilino-6-bis(2-hydroxyethyl)amino-1,3,5-triazin-2-yl)amino]stilbene-2,2′-disulfonic acid (Tinopal UNPA-GX from Ciba), 4,4′-bis[anilino-6-morpholine-1,3,5-triazin-2-yl)amino]-stilbene (Tinopal AMS-GX from Ciba), disodium 4,4′-bis[(4-anilino-6-(2-hydroxyethyl)methylamino-1,3,5-triazin-2-yl)amino]stilbene-2,2′-sulfonate (Tinopal 5BM-GX from Ciba),

2,5-thiophenediylbis(5-tert-butyl-1,3-benzoxazole) (Uvitex OB from Ciba),

the anionic derivative of diaminostilbene (dispersion in water, Leucophor BSB liquid from Clariant), and

optical brightener lakes (Covazur range from Wackherr).

As fluorescent dyes and, when they are present in the composition, optical brighteners that are useful in the present invention, mention may also be made of:

the naphthalimides of formula:

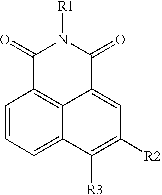

wherein R1, R2 and R3, independently of each other, are chosen from hydrogen atoms; halogen atoms; C6-C30 aryl groups; hydroxyl groups; cyano groups; nitro groups; sulfo groups; amino groups; acylamino groups; di(C1-C6)alkylamino groups; dihydroxy(C1-C6) alkylamino groups; (C1-C6)alkylhydroxy(C1-C6)alkylamino groups; (C1-C6)alkoxy groups; (C1-C6)alkoxycarbonyl groups; carboxy(C1-C6)alkoxy groups; piperidinosulfonyl groups; pyrrolidino groups; (C1-C6)alkylhalo(C1-C6)alkylamino groups; benzoyl(C1-C6)alkyl groups; vinyl groups; formyl groups; C6-C30 aryl radicals optionally substituted with at least one group chosen from hydroxyl groups, linear, branched or cyclic C1-C6 alkoxy groups, and linear, branched or cyclic alkyl groups comprising from 1 to 22 carbon atoms, optionally substituted with at least one group chosen from hydroxyl, amino and C1-C6 alkoxy groups; and linear, branched or cyclic alkyl radicals comprising from 1 to 22 carbon atoms, for example from 1 to 6 carbon atoms, optionally substituted with at least one group chosen from hydroxyl groups, amino groups, linear, branched or cyclic C1-C6 alkoxy groups, optionally substituted aryl groups, carboxyl groups, sulfo groups and halogen atoms, the alkyl radical possibly being interrupted with at least one heteroatom, such as N, S or O;

wherein the substituents R1, R2 and R3 may form, with the carbon atoms to which they are attached, a C6-C30 aromatic or non-aromatic or heterocyclic ring comprising in total from 5 to 30 ring members and from 1 to 5 heteroatoms; these rings being fused or non-fused, optionally comprising a carbonyl group, and being unsubstituted or substituted with at least one group chosen from C1-C4 alkyl, (C1-C4)alkoxy(C1-C4)alkyl, amino, di(C1-C4)alkylamino, halogen, phenyl, carboxyl and tri(C1-C4)alkylammonio(C1-C4)alkyl groups.

Mention may be made, for example, of the following compound:

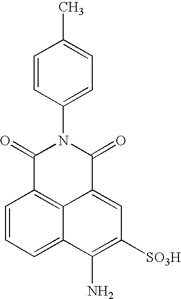

Brillant sulfoflavin FF, C.I. 56205;

The diketopyrrolopyrroles of formula:

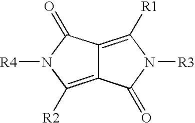

wherein R1, R2, R3 and R4, independently of each other, are chosen from hydrogen atoms; halogen atoms; from C6-C30 aryl groups; hydroxyl groups; cyano groups; nitro groups; sulfo groups; amino groups; acylamino groups; di(C1-C6)alkylamino groups; dihydroxy(C1-C6) alkylamino groups; (C1-C6)alkylhydroxy(C1-C6)alkylamino groups; (C1-C6)alkoxy groups; (C1-C6)alkoxycarbonyl groups; carboxy(C1-C6)alkoxy groups; piperidinosulfonyl groups; pyrrolidino groups; (C1-C6)alkylhalo(C1-C6)alkylamino groups; benzoyl(C1-C6)alkyl groups; vinyl groups; formyl groups; C6-C30 aryl radicals optionally substituted with at least one group chosen from hydroxyl groups, linear, branched or cyclic C1-C6 alkoxy groups, and linear, branched or cyclic alkyl groups comprising from 1 to 22 carbon atoms, optionally substituted with at least one group chosen form hydroxyl, amino and C1-C6 alkoxy groups; and linear, branched or cyclic alkyl radicals comprising from 1 to 22 carbon atoms, for example from 1 to 6 carbon atoms, optionally substituted with at least one group chosen from hydroxyl groups, amino groups, linear, branched or cyclic C1-C6 alkoxy groups, optionally substituted aryl groups, carboxyl groups, sulfo groups and halogen atoms, the alkyl radical possibly being interrupted with at least one heteroatom, such as nitrogen, sulfur or oxygen.

Mention may be made, for example, of the following compound:

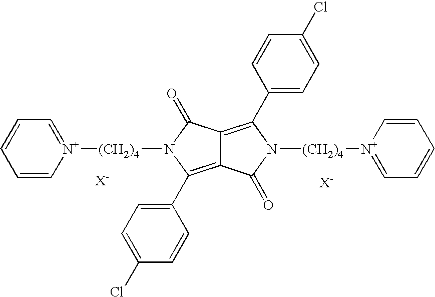

wherein X⁻ is an anion defined as above;

the dicyanopyrazine derivatives of formulae:

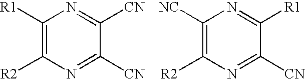

wherein R1 and R2, independently of each other, are chosen from hydrogen atoms; halogen atoms; C6-C30 aryl groups; hydroxyl groups; cyano groups; nitro groups; sulfo groups; amino groups; acylamino groups; di(C1-C6)alkylamino groups; dihydroxy(C1-C6) alkylamino groups; (C1-C6)alkylhydroxy(C1-C6)alkylamino groups; (C1-C6)alkoxy groups; (C1-C6)alkoxycarbonyl groups; carboxy(C1-C6)alkoxy groups; piperidinosulfonyl groups; pyrrolidino groups; (C1-C6)alkylhalo(C1-C6)alkylamino groups; benzoyl(C1-C6)alkyl groups; vinyl groups; formyl groups; C6-C30 aryl radicals optionally substituted with at least one group chosen from hydroxyl groups, linear, branched or cyclic C1-C6 alkoxy groups, and linear, branched or cyclic alkyl groups comprising from 1 to 22 carbon atoms, optionally substituted with at least one group chosen from hydroxyl, amino and C1-C6 alkoxy groups; and linear, branched or cyclic alkyl radicals comprising from 1 to 22 carbon atoms, for example from 1 to 6 carbon atoms, optionally substituted with at least one group chosen from hydroxyl groups, amino groups, linear, branched or cyclic from C1-C6 alkoxy groups, optionally substituted aryl groups, carboxyl groups, sulfo groups and halogen atoms, the alkyl radical possibly being interrupted with at least one heteroatom, such as nitrogen, sulfur and oxygen;
wherein two of the substituents R1 and R2 may form, with the carbon atoms to which they are attached, a C6-C30 aromatic or non-aromatic or heterocyclic ring comprising in total from 5 to 30 ring members and from 1 to 5 heteroatoms; these rings being fused or non-fused, optionally comprising at least one carbonyl group, and being unsubstituted or substituted with at least one group chosen from C1-C4 alkyl, (C1-C4)alkoxy(C1-C4)alkyl, amino, di(C1-C4)alkylamino, halogen, phenyl, carboxyl and tri(C1-C4)alkylammonio(C1-C4)alkyl groups;
and wherein two of the substituents R2 and R3 may form, with the carbon atoms to which they are attached, a C6-C30 aromatic or heterocyclic nucleus comprising in total from 5 to 30 ring members and from 1 to 5 heteroatoms; this ring being fused or non-fused, this ring and the possible fused ring being unsubstituted or substituted with at least one group chosen from C1-C4 alkyl, (C1-C4)alkoxy(C1-C4)alkyl, amino, di(C1-C4)alkylamino, halogen, phenyl, carboxyl and tri(C1-C4)alkylammonio(C1-C4)alkyl groups;

coumarin derivatives such as the compounds corresponding to the following formulae:

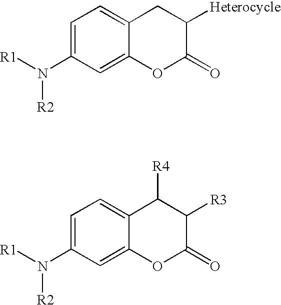

wherein the heterocycle is chosen from furan, thiophene, 2H-pyrrole, 2-pyrroline, pyrrolidine, 1,3-dioxolane, oxazole, thiazole, imidazole, 2-imidazoline, imidazoline, pyrazole, 2-pyrazoline, pyrazolidine, isoxazole, isothiazole, 1,2,3-oxadiazole, 1,2,3-triazole, 1,3,4-thiadiazole, 2H-pyran, 4H-pyran, pyridine, piperidine, 1,4-dioxane, morpholine, 1,4-dithiane, thiomorpholine, pyradizine, pyrimidine, pyrazine, piperazine, 1,3,5-triazine, 1,3,5-trithiane, indolizine, indole, isoindole, 3H-indole, indoline, benzo[b]furan, benzo[b]thiophene, 1H-indazole, benzimidazole, benzothiazole, purine, 4H-quinolizine, quinoline, isoquinoline, cinnoline, phthalazine, quinazoline, quinoxaline, 1,8-naphthyridine, pteridine, quinuclidine, carbazole, acridine, phenazine, phenothiazine, phenoxazine, indene, naphthalene, azulene, fluorene, anthracene, norbornane, and adamantane; and R1, R2, R3 and R4, independently of each other, are chosen from hydrogen atoms; halogen atoms; C6-C30 aryl groups; hydroxyl groups; cyano groups; nitro groups; sulfo groups; amino groups; acylamino groups; di(C1-C6)alkylamino groups; dihydroxy(C1-C6) alkylamino groups; (C1-C6)alkylhydroxy(C1-C6)alkylamino groups; (C1-C6)alkoxy groups; (C1-C6)alkoxycarbonyl groups; carboxy(C1-C6)alkoxy groups; piperidinosulfonyl groups; pyrrolidino groups; (C1-C6)alkylhalo(C1-C6)alkylamino groups; benzoyl(C1-C6)alkyl groups; vinyl groups; formyl groups; C6-C30 aryl radicals optionally substituted with at least one group chosen from hydroxyl groups, linear, branched or cyclic C1-C6 alkoxy groups, and linear, branched or cyclic alkyl groups comprising from 1 to 22 carbon atoms, optionally substituted with at least one hydroxyl, amino or C1-C6 alkoxy group; and linear, branched or cyclic alkyl radicals comprising from 1 to 22 carbon atoms, for example from 1 to 6 carbon atoms, optionally substituted with at least one group chosen from hydroxyl groups, amino groups, linear, branched or cyclic C1-C6 alkoxy groups, optionally substituted aryl groups, carboxyl groups, sulfo groups and halogen atoms, the alkyl radical possibly being interrupted with at least one heteroatom, such as a nitrogen atom, a sulphur atom or an oxygen atom; wherein the substituents R1, R2, R3 and R4 may form, with the carbon atoms to which they are attached, a C6-C30 aromatic or non-aromatic or heterocyclic ring comprising in total from 5 to 30 ring members and from 1 to 5 heteroatoms; these rings being fused or non-fused, optionally comprising a carbonyl group, and being unsubstituted or substituted with at least one group chosen from C1-C4 alkyl, (C1-C4)alkoxy(C1-C4)alkyl, amino, di(C1-C4)alkylamino, halogen, phenyl, carboxyl and tri(C1-C4)alkylammonio(C1-C4)alkyl groups; and wherein two of the substituents R3 and R4 may form, with the carbon atoms to which they are attached, a C6-C30 aromatic or heterocyclic nucleus comprising in total from 5 to 30 ring members and from 1 to 5 heteroatoms; the ring being fused or non-fused, the ring and the possible fused ring being unsubstituted or substituted with at least one group chosen from. C1-C4 alkyl, (C1-C4)alkoxy(C1-C4)alkyl, amino, di(C1-C4)alkylamino, halogen, phenyl, carboxyl and tri(C1-C4)alkylammonio(C1-C4)alkyl groups.

By way of example, mention may be made of:

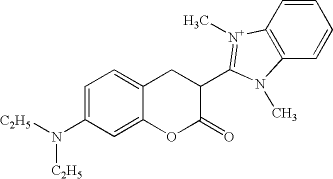

C.I. Basic yellow 40;

naphtholactam derivatives such as:

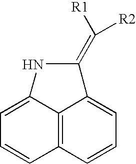

wherein R1 and R2 are as defined as above.

An example that may be mentioned is the following compound:

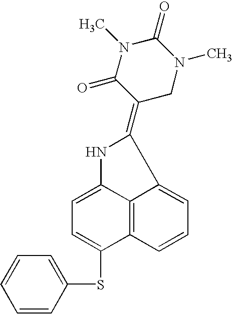

### Disperse dye 28;

azlactone derivatives:

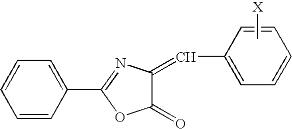

wherein X has the same definition as R1 described above.

An example that may be mentioned is the following compound:

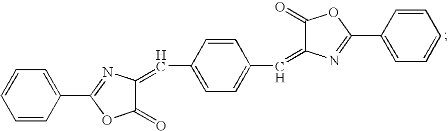

methine derivatives such as:

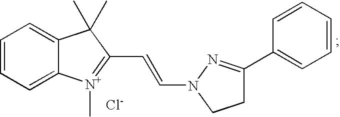

oxazine and thiazine derivatives such as:

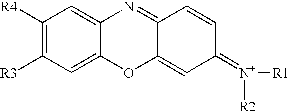

wherein R1, R2, R3 and R4 are as defined above. An example that may be mentioned is:

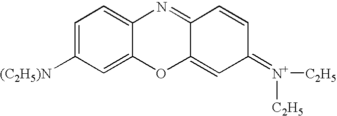

### Basic Blue 3, C.I. 51004;

1,4-distyrylbenzene derivatives of formula:

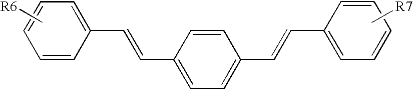

wherein R6 and R7, independently of each other, are chosen from hydrogen atoms; halogen atoms; C6-C30 aryl groups; hydroxyl groups; cyano groups; nitro groups; sulfo groups; amino groups; acylamino groups; di(C1-C6)alkylamino groups; dihydroxy(C1-C6)-alkylamino groups; (C1-C6)alkylhydroxy(C1-C6)alkylamino groups; (C1-C6)alkoxy groups; (C1-C6)alkoxycarbonyl groups; carboxy(C1-C6)alkoxy groups; piperidinosulfonyl groups; pyrrolidino groups; (C1-C6)alkylhalo(C1-C6)alkylamino groups; benzoyl(C1-C6)alkyl groups; vinyl groups; formyl groups; C6-C30 aryl radicals optionally substituted with at least one group chosen from hydroxyl groups, linear, branched or cyclic C1-C6 alkoxy groups, and linear, branched or cyclic alkyl groups comprising from 1 to 22 carbon atoms, optionally substituted with at least one hydroxyl, amino or C1-C6 alkoxy group; and linear, branched or cyclic alkyl radicals comprising from 1 to 22 carbon atoms, for example from 1 to 6 carbon atoms, optionally substituted with at least one group chosen from hydroxyl groups, amino groups, linear, branched or cyclic C1-C6 alkoxy groups, optionally substituted aryl groups, carboxyl groups, sulfo groups and halogen atoms, the alkyl radical possibly being interrupted with at least one heteroatom, such as nitrogen, sulfur or oxygen;

the 4,4′-distyrylbiphenyl derivatives of formula:

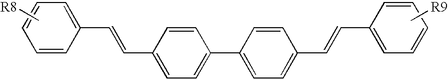

wherein R8 and R9, independently of each other, are chosen from hydrogen atoms; halogen atoms; C6-C30 aryl groups; hydroxyl groups; cyano groups; nitro groups; sulfo groups; amino groups; acylamino groups; di(C1-C6)alkylamino groups; dihydroxy(C1-C6)-alkylamino groups; (C1-C6)alkylhydroxy(C1-C6)alkylamino groups; (C1-C6)alkoxy groups; (C1-C6)alkoxycarbonyl groups; carboxy(C1-C6)alkoxy groups; piperidinosulfonyl groups; pyrrolidino groups; (C1-C6)alkylhalo(C1-C6)alkylamino groups; benzoyl(C1-C6)alkyl groups; vinyl groups; formyl groups; C6-C30 aryl radicals optionally substituted with at least one group chosen from hydroxyl groups, linear, branched or cyclic C1-C6 alkoxy groups, and linear, branched or cyclic alkyl groups comprising from 1 to 22 carbon atoms, optionally substituted with at least one group chosen from hydroxyl, amino and C1-C6 alkoxy groups; and linear, branched or cyclic alkyl radicals comprising from 1 to 22 carbon atoms, for example from 1 to 6 carbon atoms, optionally substituted with at least one group chosen from hydroxyl groups, amino groups, linear, branched or cyclic C1-C6 alkoxy groups, optionally substituted aryl groups, carboxyl groups, sulfo groups and halogen atoms, the alkyl radical possibly being interrupted with at least one heteroatom, such as nitrogen, sulfur or oxygen;

the triazinylaminostilbene derivatives of formula:

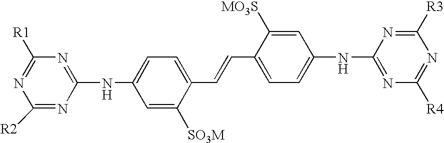

wherein R1, R2, R3 and R4, independently of each other, are chosen from hydrogen atoms; halogen atoms; C6-C30 aryl groups; hydroxyl groups; cyano groups; nitro groups; sulfo groups; amino groups; acylamino groups; di(C1-C6)alkylamino groups; dihydroxy(C1-C6)alkylamino groups; (C1-C6)alkylhydroxy(C1-C6)alkylamino groups; (C1-C6)alkoxy groups; (C1-C6)alkoxycarbonyl groups; carboxy(C1-C6)alkoxy groups; piperidinosulfonyl groups; pyrrolidino groups; (C1-C6)alkylhalo(C1-C6)alkylamino groups; benzoyl-(C1-C6)alkyl groups; vinyl groups; formyl groups; C6-C30 aryl radicals optionally substituted with at least one group chosen from hydroxyl groups, linear, branched or cyclic C1-C6 alkoxy groups, and linear, branched or cyclic alkyl groups comprising from 1 to 22 carbon atoms, optionally substituted with at least one group chosen from hydroxyl, amino and C1-C6 alkoxy groups; and linear, branched or cyclic alkyl radicals comprising from 1 to 22 carbon atoms, for example from 1 to 6 carbon atoms, optionally substituted with at least one group chosen from hydroxyl groups, amino groups, linear, branched or cyclic C1-C6 alkoxy groups, optionally substituted aryl groups, carboxyl groups, sulfo groups and halogen atoms, the alkyl radical possibly being interrupted with at least one heteroatom, such as nitrogen, sulfur or oxygen; and wherein M is a monovalent or divalent cation derived from the family of alkali metals or alkaline-earth metals, for instance sodium, potassium and calcium ions;

the bis(benzoxazole) derivatives of formula:

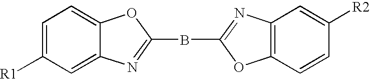

wherein R1 and R2, independently of each other, are chosen from hydrogen atoms; halogen atoms; C6-C30 aryl groups; hydroxyl groups; cyano groups; nitro groups; sulfo groups; amino groups; acylamino groups; di(C1-C6)alkylamino groups; dihydroxy(C1-C6)-alkylamino groups; (C1-C6)alkylhydroxy(C1-C6)alkylamino groups; (C1-C6)alkoxy groups; (C1-C6)alkoxycarbonyl groups; carboxy(C1-C6)alkoxy groups; piperidinosulfonyl groups; pyrrolidino groups; (C1-C6)alkylhalo(C1-C6)alkylamino groups; benzoyl(C1-C6)alkyl groups; vinyl groups; formyl groups; C6-C30 aryl radicals optionally substituted with at least one group chosen from hydroxyl groups, linear, branched or cyclic C1-C6 alkoxy groups, and linear, branched or cyclic alkyl groups comprising from 1 to 22 carbon atoms, optionally substituted with at least one group chosen from hydroxyl, amino and C1-C6 alkoxy groups; and linear, branched or cyclic alkyl radicals comprising from 1 to 22 carbon atoms, for example from 1 to 6 carbon atoms, optionally substituted with at least one group chosen from hydroxyl groups, amino groups, linear, branched or cyclic C1-C6 alkoxy groups, optionally substituted aryl groups, carboxyl groups, sulfo groups and halogen atoms, the alkyl radical possibly being interrupted with at least one heteroatom, such as nitrogen, sulfur or oxygen; and

B is chosen from:

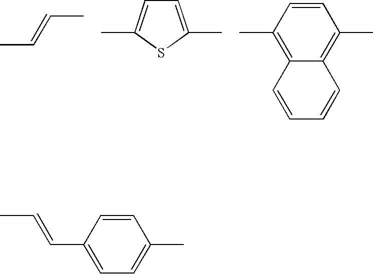

cationic or non-cationic bis(benzimidazoles); and

anionic or non-anionic 1,3-diphenyl-2-pyrazolines, for example:

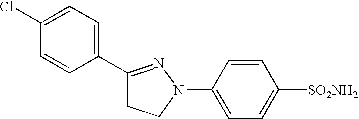

Non-limiting mention may be made of the compounds in the following publication: “Selective topochemicalphotoreaction of crystallized 2,3-(phenyletheny)-4,5-dicyanopyrazine” by Kim, Jae Hong; Matsuoka Masaru, Chem. Lett. (1999), (2), 143-144.

In at least one embodiment, non-limiting mention may be made of:

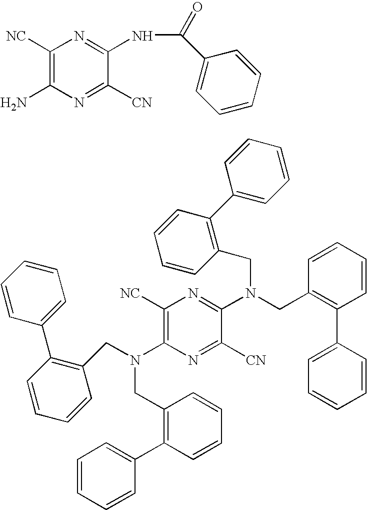

The amount of soluble fluorescent compounds that are useful in the present disclosure may vary within very wide ranges. By way of example, the fluorescent compounds may be present in an amount ranging from 0.001% to 40% by weight relative to the total weight of the composition, for example from 0.01% and 20% or from 0.1% to 10% by weight relative to the total weight of the composition.

### III) Organic Solvents

As used herein, the term “organic solvent” means an organic substance that is liquid at a temperature of 25° C. and at atmospheric pressure (760 mmHg), which is capable of dissolving another substance without chemically modifying it.

In one embodiment of the present disclosure, the at least one organic solvent is chosen, for example, from aromatic alcohols such as benzyl alcohol, phenoxy-ethanol and phenylethyl alcohol; liquid fatty alcohols, for example of C₁₀-C₃₀; C₁-C₆ alkanols such as ethanol, isopropanol, n-propanol, butanol, n-pentanol, 1,2-propanediol, 1,3-propanediol, 1-methoxy-2-propanol, 1-ethoxy-2-propanediol, 1,3- and 1,4-butanediol and 1,2-hexanediol; polyols and polyol ethers comprising a free-OH function such as 2-butoxyethanol, propylene glycol, propylene glycol monomethyl ether, diethylene glycol monoethyl ether and monomethyl ether, ethylene glycol monoethyl ether, ethylene glycol monobutyl ether, neopentyl glycol, isoprene glycol, glycerol, glycol, dipropylene glycol, butylene glycol and butyldiglycol; volatile silicones such as short-chain linear silicones such as hexamethyldisiloxane or octamethyltrisiloxane, cyclic silicones such as octamethylcyclotetrasiloxane, decamethylcyclopentasiloxane or dodecamethylcyclohexasiloxane, polydimethylsiloxanes optionally modified with alkyl and/or amine and/or imine and/or fluoroalkyl and/or carboxylic and/or betaine and/or quaternary ammonium functions; liquid modified polydimethylsiloxanes; mineral, organic or plant oils; alkanes, for example C₅-C₁₀ alkanes; liquid fatty acids; and liquid fatty esters, for example liquid fatty alkyl benzoates or salicylates.

In one embodiment, the at least one organic solvent is chosen from organic oils; silicones such as volatile silicones, amino or non-amino silicone gums or oils, and mixtures thereof; mineral oils; plant oils such as olive oil, castor oil, rapeseed oil, coconut oil, wheatgerm oil, apricot kernel oil, avocado oil, macadamia oil, apricot oil, safflower oil, candlenut oil, camellina oil, tamanu oil, lemon oil or organic compounds such as C₅-C₂₀ alkanes, acetone, methyl ethyl ketone, esters of liquid C₁-C₂₀ acids and of C₁-C₈ alcohols such as methyl acetate, butyl acetate, ethyl acetate and isopropyl myristate, dimethoxyethane, diethoxyethane, liquid C₁₀-C₃₀ fatty alcohols such as oleyl alcohol, esters of fatty alcohols or of fatty acids such as C₁₀-C₃₀ fatty alkyl benzoates, and mixtures thereof; isononyl isononanoate, isostearyl malate, pentaerythrityl tetraisostearate, tridecyl trimellitate, polybutene oil, the mixture of cyclopentasiloxane (14.7% by weight)/poly-dimethylsiloxane dihydroxylated in the α and ω positions (85.3% by weight); and mixtures thereof.

According to one embodiment, the at least one organic solvent is chosen from silicones such as liquid polydimethylsiloxanes and modified liquid polydimethylsiloxanes, their viscosity at 25° C. ranging from 0.1 cSt to 1,000,000 cSt, for example from 1 cSt to 30,000 cSt.

In one embodiment, at least one of the following oils is chosen:

the mixture of α,ω-dihydroxylated polydimethyl-siloxane/cyclopentadimethylsiloxane (14.7/85.3) sold by Dow Corning under the name DC 1501 Fluid;

the mixture of α,ω-dihydroxylated polydimethylsiloxane/polydimethylsiloxane sold by Dow Corning under the name DC 1503 Fluid;

the mixture of dimethicone/cyclopentadimethylsiloxane sold by Dow Corning under the name DC 1411 Fluid or that sold by Bayer under the name SF1214;

the cyclopentadimethylsiloxane sold by Dow Corning under the name DC245 Fluid;

and mixtures thereof.

These organic solvents may serve as diluents for the polycondensation reactions.

In one embodiment, the at least one organic solvent of the composition is present in an amount ranging from 0.01% to 99% by weight relative to the total weight of the composition, for example from 50% to 99%.

Besides the at least one liquid organic solvent the composition of the disclosure may contain water. The composition of the disclosure may be anhydrous, i.e. it may contain less than 1% by weight of water relative to the total weight of the composition.

The composition of the invention may also be in the form of an emulsion and/or may be encapsulated. When the composition is an emulsion, it may comprise, for example, a dispersed or continuous phase, which may be water, C1-C4 aliphatic alcohols, or mixtures thereof and a water-insoluble organic phase.

The composition in accordance with the disclosure may also comprise the compounds of the present disclosure, for example the soluble fluorescent compounds, the pigments and/or the optional organic solvents; at least one agent usually used in cosmetics chosen, for example, from reducing agents, fatty substances, plasticizers, softeners, antifoams, moisturizers, pigments, clays, mineral fillers, UV-screening agents, mineral colloids, peptizers, fragrances, preserving agents, anionic, cationic, nonionic or amphoteric surfactants, fixing or non-fixing polymers, polyols, proteins, vitamins, direct dyes, oxidation dyes, nacres, propellants, mineral or organic thickeners such as benzylidenesorbitol and N-acylamino acids, oxyethylenated or non-oxyethylenated waxes, paraffins, solid acids: stearic acid, lauric acid, C₁₀-C₃₀ fatty amides such as lauric diethanolamide, and fatty alcohol esters, and mixtures thereof.

This composition may be in various forms, such as in the form of lotions, sprays or mousses, and may be applied in the form of a shampoo or a hair conditioner.

In the case of sprays, the composition of the disclosure may contain a propellant, wherein the propellant may comprise at least one compressed or liquefied gas as usually used for the preparation of aerosol compositions. For example, air, carbon dioxide, compressed nitrogen or at least one soluble gas such as dimethyl ether, halohydrocarbons (such as fluorohydrocarbons) or non-halohydrocarbons, and/or mixtures thereof may be used.

Pocket aerosols may also be used, where appropriate.

The composition of the disclosure may be applied to dry or wet keratin fibers, and may be dried in the open air or using a hairdryer or a hood. Flat tongs may be used after pre-drying the keratin fibers.

The process according to the present disclosure comprises applying to the keratin fibers at least one compound X and at least one compound Y which, when placed in contact with each other, react together via a hydrosilylation reaction optionally in the presence of a hydrosilylation catalyst, a condensation reaction, or a crosslinking reaction in the presence of a peroxide; and also applying at least one fluorescent dye and optionally at least one optical brightener as defined above.

The at least one compound X and the at least one compound Y may be applied to the keratin fibers using several compositions comprising compounds X and Y, the at least one fluorescent dye and the optional at least one optical brightener, alone or as a mixture, or using a single composition comprising the at least one compound X, the at least one compound Y, the at least one fluorescent dye and, optionally, at least one optical brightener and/or at least one organic solvent.

According to one embodiment, compound X, compound Y, the fluorescent dye(s) and optional optical brightener(s) and the organic solvent(s) are applied independently of each other.

According to another embodiment of the present disclosure, a composition (C) comprising the at least one compound X, the at least one compound Y, and the at least one fluorescent dye, and optionally at least one optical brightener is applied to the keratin fibers optionally in the presence of at least one solvent.

According to another embodiment of the present disclosure, a composition (D) comprising the at least one fluorescent dye and optionally at least one optical brightener, and a composition (E) comprising the at least one compound X and the at least one compound Y are applied to the keratin fibers, at least one organic solvent being optionally present in composition (D) and/or composition (E), compositions (D) and (E) being applied in any order.

According to another embodiment of the present disclosure, a composition (D) comprising at least one pigment, at least one fluorescent dye and optionally at least one optical brightener, a composition (A) comprising the at least one compound X, and a composition (B) comprising the at least one compound Y are applied to the keratin fibers, at least one organic solvent being optionally present in composition (D) and/or composition (A) and/or composition (B), compositions (A), (B) and (D) being applied in any order.

According to another embodiment of the present disclosure, a composition (F) comprising the at least one compound X and the at least one fluorescent dye and optionally at least one optical brightener, and a composition (B) comprising the at least one compound Y are applied to the keratin fibers, at least one organic solvent being optionally present in composition (B) and/or composition (F), compositions (B) and (F) being applied in any order.

According to another embodiment of the present disclosure, a composition (A) comprising at least one compound X, and a composition (G) comprising at least one compound Y and the at least one fluorescent dye, together with the optional at least one optical brightener and/or an optional pigment are applied to the keratin fibers, at least one organic solvent being optionally present in composition (A) and/or composition (G), compositions (A) and (G) being applied in any order. According to at least one embodiment, the composition comprising the at least one pigment, fluorescent dye or optical brightener is applied before the composition(s) comprising at least one compound X and/or the at least one compound Y.

In one embodiment, when compounds X and Y are capable of reacting together via a crosslinking reaction, at least one peroxide as described above is applied to the keratin fibers. The at least one peroxide may be present in one or other of the composition(s) mentioned or in an additional composition.

According to another embodiment of the disclosure, at least one catalyst as defined above is applied to the keratin fibers to activate the reaction between at least one compound X and at least one compound Y.

For example, at least one catalyst may be present in one or other or in several of the compositions applied to the keratin fibers or in an additional composition, in which case the various compositions may be applied to the keratin fibers in any order.

In one embodiment, at least one catalyst is chosen from those described previously.

In one embodiment, when at least one catalyst and/or at least one peroxide is applied, the at least one compound X and/or the at least one compound Y, the at least one catalyst and/or the at least one peroxide are not simultaneously stored in the same composition. In a further embodiment, they may be mixed together at the time of use.

According to another embodiment of the disclosure, at least one additional reactive compound as defined above is applied to the keratin fibers.

For example, the at least one additional reactive compound may be present in one or the other or in several of the compositions applied to the keratin fibers or in an additional composition, in which case the various compositions may be applied to the keratin fibers in any order.

In one embodiment, at least one additional reactive compound is chosen from those described previously.

In one embodiment, the various compositions used in the process in accordance with the disclosure may be applied to dry or wet hair.

In one embodiment, intermediate drying and/or rinsing may be performed between each application.

In one embodiment, a composition used in the process in accordance with the disclosure comprises a cosmetically acceptable medium, which conveys the at least one compound X and/or the at least one compound Y, and is chosen such that compounds X and Y are able to react with each other via a hydrosilylation, condensation or crosslinking reaction in the presence of at least one peroxide after the application of the cosmetic composition to the hair.

In one embodiment, the deposit thus formed has the advantage of having an expected low solubility. In another embodiment, it shows good affinity for the surface of keratin fibers, which ensures better remanence of the deposit as a whole.

In another embodiment, when compounds X and Y are applied separately, the deposit in coats obtained may also preserve the cosmetic or optical properties of the compound that constitutes the top part of the deposit.

In a further embodiment according to the same processes, it is possible to produce multiple superpositions of coats of compounds X and Y alternately or otherwise to achieve the desired type of deposit (in terms of chemical nature, mechanical strength, thickness, appearance, feel, etc.).

In another embodiment the disclosure also relates to a dyeing kit comprising at least two compositions (A) and (B) conditioned separately, the kit comprising i) at least one compound X, ii) at least one compound Y, it being understood that composition (A) and/or (B) comprise(s) at least one fluorescent dye and optionally at least one optical brightener; and iii) optionally at least one catalyst or at least one peroxide; wherein the at least one catalyst, when it is present, or the at least one peroxide are not simultaneously present in the same composition; wherein the compounds X and Y, when placed in contact with each other, react together via a hydrosilylation reaction, or via a condensation reaction or via a crosslinking reaction in the presence of a peroxide.

In another embodiment, the kit comprises a multi-compartment device with a first compartment comprising a composition (A) comprising at least one compound X, a second compartment comprising a composition (B) comprising at least one compound Y, and a third compartment comprising a composition (D) comprising at least one fluorescent dye and an optional optical brightener.

According to one embodiment, the kit comprises two compartments, the first compartment comprising a composition (C) comprising at least one compound X, at least one compound Y and at least one fluorescent dye and optional optical brightener, and a second compartment comprising at least one catalyst or at least one peroxide.

In another embodiment, the kit comprises a multi-compartment device comprising a first compartment of a composition (F) comprising at least one compound X and at least one fluorescent dye and optional optical brightener optionally in the presence of a solvent, and another compartment of a composition (B) comprising at least one compound Y, and optionally a third compartment comprising at least one catalyst.

In another embodiment, the kit comprises a first compartment of a composition (G) comprising at least one compound Y and at least one fluorescent dye and optional optical brightener optionally in the presence of a solvent, and another compartment of a composition (A) comprising at least one compound X, and optionally a third compartment comprising at least one catalyst.

Other than in the examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the disclosure are approximations, unless otherwise indicated the numerical values set forth in the specific examples are reported as precisely as possible. Any numerical value, however, inherently contains certain errors necessarily resulting from the standard deviation found in their respective testing measurements.

By way of non-limiting illustration, concrete examples of certain embodiments of the present disclosure are given below.

Unless otherwise mentioned, the amounts indicated are expressed as mass percentages.

### EXAMPLES

### Condensation

Examples of combinations of compounds X and Y comprising alkoxysilane groups and reacting via condensation, include, but are not limited to, the following compositions comprising mixtures A′ and B′ prepared by the company Dow Corning.

### With a Fluorescent Dye:

| Composition 1 | in g |
|---|---|
| Cyclopentadimethylsiloxane sold by Dow Corning under the | 73.34 |
| name DC245 Fluid | |
| Mixture of polydimethylsiloxane containing methoxy groups, | 15.15 |
| hexamethyldisiloxane and treated silica, sold by Dow Corning: | |
| Mixture A′ | |
| Sunbrite-SG2515 Yellow orange pigments from SunChemical | 10 |

wherein Mixture A′:

| Ingredient (INCI name) | CAS No. | Contents (%) | Function |
|---|---|---|---|
| bis-Trimethoxysiloxyethyl | PMN87176 | 25-45 | Polymer |
| Tetramethyldisiloxyethyl | | | |
| Dimethicone* | | | |
| Silica Silylate | 68909-20-6 | 5-20 | Filler |
| Disiloxane | 107-46-0 | 30-70 | Solvent |

*corresponds to identical compounds X and Y

| Composition 2 | in g |
|---|---|
| Mixture of hexamethyldisiloxane and titanium catalyst sold by | 1.51 |
| Dow Corning: Mixture B′ | |

wherein Mixture B′:

| Ingredient (INCI name) | CAS No. | Contents (%) | Function |
|---|---|---|---|
| Disiloxane | 107-46-0 | 80-99 | Solvent |
| Tetra T Butyl Titanate | — | 1-20 | Catalyst |

The two compositions 1 and 2 above were mixed together at the time of use to obtain 100 g of a mixture. 0.5 g of this mixture was applied to a lock of 1 g of clean, wet natural hair with a tone depth of 4. After a leave-on time of one hour, the lock was dried with a hairdryer for 2 minutes. A dyed lock whose hairs were individualized was obtained. The color was remanent.

### With an Optical Brightener:

| Composition 3 | in g |
|---|---|
| Cyclopentadimethylsiloxane sold by Dow Corning under the | 73.34 |
| name DC245 Fluid | |
| Mixture of polydimethylsiloxane comprising methoxy groups, | 15.15 |
| hexamethyldisiloxane and treated silica, sold by Dow Corning: | |
| Mixture A′ | |
| Diethylaminocoumarin from Ciba | 10 |

| Composition 2 | in g |
|---|---|
| Mixture of hexamethyldisiloxane and titanium catalyst sold by | 1.51 |
| Dow Corning: Mixture B′ | |

wherein mixtures A′ and B′ are as defined above.

The two compositions above were mixed together at the time of use to obtain 100 g of a mixture. 0.5 g of this mixture was applied to a lock of 1 g of natural hair with a tone depth of 4. After a leave-on time of one hour, the lock was dried with a hairdryer for 2 minutes. A shiny lock whose hairs were individualized was obtained. The sheen was remanent.

### Hydrosilylation

Examples of combinations of compounds X and Y reacting via hydrosilylation, include, but are not limited to, compositions 4, 5 and 6, and also mixtures A and B below sold by the company Dow Corning:

With a Fluorescent Pigment:

| | % by |
|---|---|
| Composition 4 | weight |
| Cyclopentadimethylsiloxane sold by Dow Corning under the | 70 |
| name DC245 Fluid | |
| Mixture of polydimethylsiloxane comprising vinyl end groups, | 10 |
| hexamethyldisiloxane, Pt (II) catalyst and treated silica, sold by | |
| Dow Corning: Mixture A | |
| Sunbrite-SG2515 Yellow orange pigments from SunChemical | 20 |

wherein Mixture A:

| Ingredient (INCI name) | CAS No. | Contents (%) | Function |
|---|---|---|---|
| Dimethyl Siloxane, | 68083-19-2 | 55-95 | Polymer |
| Dimethylvinylsiloxy-terminated | | | |
| Silica Silylate | 68909-20-6 | 10-40 | Filler |
| 1,3-Diethenyl-1,1,3,3-Tetra- | 68478-92-2 | Trace | Catalyst |
| methyldisiloxane complexes | | | |
| Tetramethyldivinyldisiloxane | 2627-95-4 | 0.1-1 | Polymer |

| | % by |
|---|---|
| Composition 5 | weight |
| Cyclopentadimethylsiloxane sold by Dow Corning under the | 90 |
| name DC245 Fluid | |
| Mixture of polydimethylsiloxane comprising vinyl end groups, | 10 |
| methylhydrogenosiloxane (crosslinking agent) and treated silica, | |
| sold by Dow Corning: Mixture B | |

wherein Mixture B:

| Ingredient (INCI name) | CAS No. | Contents (%) | Function |
|---|---|---|---|
| Dimethyl Siloxane, | 68083-19-2 | 55-95 | Polymer |
| Dimethylvinylsiloxy- | | | |
| terminated | | | |
| Silica Silylate | 68909-20-6 | 10-40 | Filler |
| Dimethyl, Methylhydrogen | 68037-59-2 | 1-10 | Polymer |
| Siloxane, trimethylsiloxy- | | | |
| terminated | | | |

The two compositions 4 and 5 were mixed together at the time of use, in a 50/50 weight proportion. 0.5 g of this mixture was applied to a lock of 1 g of clean, wet natural hair with a tone depth of 4. After a leave-on time of one hour, the lock was dried with a hairdryer for 2 minutes. A dyed lock whose hairs were individualized was obtained. The color was remanent.

### With an Optical Brightener:

| | % by |
|---|---|
| Composition 6 | weight |
| Cyclopentadimethylsiloxane sold by Dow Corning under the | 70 |
| name DC245 Fluid | |
| Mixture of polydimethylsiloxane comprising vinyl end groups, | 10 |
| hexamethyldisiloxane, Pt (II) catalyst and treated silica, sold by | |
| Dow Corning: Mixture B | |
| Diethylaminocoumarin from Ciba | 20 |

| | % by |
|---|---|
| Composition 5 | weight |
| Cyclopentadimethylsiloxane sold by Dow Corning under the | 90 |
| name DC245 Fluid | |
| Mixture of polydimethylsiloxane comprising vinyl end groups, | 10 |
| methylhydrogenosiloxane (crosslinking agent) and treated silica, | |
| sold by Dow Corning: Mixture B | |

wherein mixtures A and B are as defined above.

The two compositions 6 and 5 were mixed together at the time of use in a 50/50 weight proportion. 0.5 g of this mixture was applied to a lock of 1 g of clean, wet natural hair with a tone depth of 4. After a leave-on time of one hour, the lock was dried with a hairdryer for 2 minutes. A shiny lock whose hairs were individualized was obtained. The sheen was remanent.

What is claimed is:

1. A composition for dyeing keratin fibers, comprising:
i) at least one compound X;
ii) at least one compound Y, and
iii) at least one fluorescent dye,
wherein at least one of compounds X or Y is a silicone compound, and
wherein the compounds X and Y, when they are placed in contact with each other, react together via:
a hydrosilylation reaction optionally in the presence of at least one hydrosilylation catalyst;
a condensation reaction; or
a crosslinking reaction in the presence of at least one peroxide.

2. A composition according to claim 1, wherein the compounds X and Y, when they are placed in contact with each other, react via hydrosilylation.

3. A composition according to claim 2, wherein compound X is chosen from silicone compounds comprising at least two unsaturated aliphatic groups pendent on the main chain or located at the ends of the main chain of the compound.

4. A composition according to claim 2, wherein compound X is chosen from polyorganosiloxanes comprising at least two unsaturated aliphatic groups bonded to a silicon atom.

5. A composition according to claim 2, wherein compound X is chosen from polyorganosiloxanes comprising siloxane units of formula:
$${R}_{m} {R}^{′} {SiO}_{\frac{( 3 - m )}{2}} \qquad ( I )$$
wherein:
R is a linear or cyclic monovalent hydrocarbon-based group comprising from 1 to 30 carbon atoms,
m is equal to 1 or 2, and
R′ is chosen from an unsaturated aliphatic hydrocarbon-based group comprising from 2 to 10 carbon atoms; and an unsaturated cyclic hydrocarbon-based group comprising from 5 to 8 carbon atoms.

6. A composition according to claim 5, wherein R is chosen from an alkyl radical comprising from 1 to 10 carbon atoms and a phenyl group and R′ is chosen from vinyl, allyl and hexenyl groups, and mixtures thereof.

7. A composition according to claim 5, wherein the polyorganosiloxanes further comprise units of formula:
$${R}_{n} {SiO}_{\frac{( 4 - n )}{2}} \qquad ( II )$$
wherein R is chosen from an alkyl radical comprising from 1 to 10 carbon atoms and a phenyl group, and n is equal to 1, 2 or 3.

8. A composition according to claim 2, wherein compound X is chosen from organic oligomers or polymers and organic/silicone hybrid oligomers or polymers, wherein the oligomers or polymers bear at least two reactive unsaturated aliphatic groups, and mixtures thereof.

9. A composition according to claim 8, wherein compound X is chosen from vinyl, (meth)acrylic, polyester, polyurethane and/or polyurea, polyether, perfluoropolyether and polyolefin polymers or oligomers, and organic hyperbranched dendrimers or polymers, wherein the polymers bear at least 2 reactive unsaturated aliphatic groups, and mixtures thereof.

10. A composition according to claim 2, wherein compound Y is chosen from polyorganosiloxanes comprising at least two free Si—H groups.

11. A composition according to claim 2, wherein compound Y is chosen from polyorganosiloxanes comprising alkylhydrogenosiloxane units having the following formula:
$${R}_{p} {H SiO}_{\frac{( 3 - p )}{2}} \qquad ( III )$$
wherein:
R is a linear or cyclic monovalent hydrocarbon-based group comprising from 1 to 30 carbon atoms; and
p is equal to 1 or 2.

12. A composition according to claim 11, wherein R is a methyl group.

13. A composition according to claim 11, wherein polyorganosiloxane Y comprises at least one end group of formula (CH₃)₃SiO_{1/2}.

14. A composition according to claim 2, further comprising at least one additional reactive compound comprising at least two unsaturated aliphatic groups.

15. A composition according to claim 2, further comprising at least one platinum-based or tin-based catalyst.

16. A composition according to claim 15, wherein the at least one catalyst is present in an amount ranging from 0.0001% to 20% by weight relative to the total weight of the composition.

17. A composition according to claim 1, wherein compound X is a polydimethylsiloxane comprising vinyl end groups and compound Y is methylhydrogenosiloxane.

18. A composition according to claim 1, wherein compounds X and Y, when placed in contact with each other, react via condensation.

19. A composition according to claim 18, wherein compounds X and/or Y, which may be identical or different, are chosen from compounds comprising at least two alkoxysilane groups and/or two silanol groups on the side and/or at the end of the chain.

20. A composition according to claim 19, wherein compounds X and/or Y, which may be identical or different, predominantly comprise units of formula:
R⁹ₛSiO₍₄₋ₛ₎/2, (IV)
wherein each R⁹ is independently a radical chosen from alkyl groups comprising from 1 to 6 carbon atoms, phenyl groups and fluoroalkyl groups, and s is equal to 0, 1, 2 or 3.

21. A composition according to claim 20, wherein compounds X and/or Y, which may be identical or different, are polyorganosiloxanes comprising units of formula:
(R⁹₂SiO₂)_{f}— (V)
wherein R⁹ is independently a radical chosen from alkyl groups comprising from 1 to 6 carbon atoms, phenyl groups and fluoroalkyl groups, and f is such that the polymer has a viscosity at 25° C. ranging from 0.5 to 3000 Pa·s.

22. A composition according to claim 20, wherein compounds X and/or Y, which may be identical or different, are polyorganosiloxanes comprising at least 2 trialkoxysilane end groups per polymer molecule, the groups having the following formula:
ZSiR¹ₓ(OR)₃₋ₓ, (V)
wherein:
R is independently chosen from methyl, ethyl, n-propyl, isopropyl, n-butyl, sec-butyl and isobutyl groups,
R¹ is chosen from methyl and ethyl groups,
x is equal to 0 or 1, and
Z is chosen from: divalent hydrocarbon-based groups not comprising any ethylenic unsaturation and comprising from 1 to 18 carbon atoms, combinations of divalent hydrocarbon-based radicals and of siloxane segments of formula (IX) below: 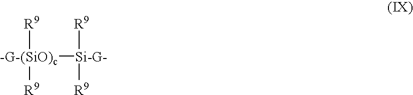
wherein
R⁹ is independently a radical chosen from alkyl groups comprising from 1 to 6 carbon atoms, phenyl groups and fluoroalkyl groups,
G is a divalent hydrocarbon-based radical not comprising any ethylenic unsaturation and comprises from 1 to 18 carbon atoms and
c is an integer ranging from 1 to 6.

23. A composition according to claim 19, wherein compounds X and/or Y, which may be identical or different, are chosen from polyorganosiloxanes chosen from polymers of formula: 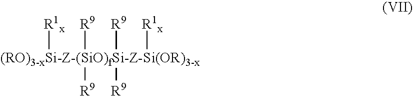
wherein
R is independently chosen from methyl, ethyl, n-propyl, isopropyl, n-butyl, sec-butyl and isobutyl groups,
R¹ is chosen from methyl and ethyl groups,
x is equal to 0 or 1,
f is such that the polymer has a viscosity at 25° C. ranging from 0.5 to 3000 Pa·s. and
Z is chosen from: divalent hydrocarbon-based groups not comprising any ethylenic unsaturation and comprising from 1 to 18 carbon atoms, combinations of divalent hydrocarbon-based radicals and of siloxane segments of formula (IX) below: 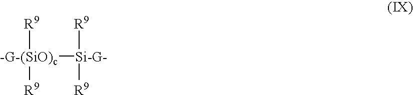
wherein
R⁹ is independently chosen from alkyl groups comprising from 1 to 6 carbon atoms, phenyl groups and fluoroalkyl groups,
G is a divalent hydrocarbon-based radical not comprising any ethylenic unsaturation and comprises from 1 to 18 carbon atoms and
c is an integer ranging from 1 to 6.

24. A composition according to claim 18, wherein
compound X is chosen from organic oligomers or polymers and organic/silicone hybrid oligomers or polymers, wherein the polymers or oligomers comprise at least two alkoxysilane groups, and
compound Y is chosen from polyorganosiloxanes.

25. A composition according to claim 18, further comprising at least one titanium-based catalyst.

26. A composition according to claim 25, wherein the at least one catalyst is present in an amount ranging from 0.0001% to 20% by weight relative to the total weight of the composition.

27. A composition according to claim 26, wherein X and Y represent a mixture of methoxysilane groups comprising polydimethylsiloxane.

28. A composition according to claim 1, wherein compounds X and Y, when they are placed in contact with each other, react via crosslinking in the presence of peroxide.

29. A composition according to claim 1, further comprising at least one filler chosen from silica and surface-treated silica.

30. A composition according to claim 1, wherein the at least one compound X has a weight-average molecular mass (Mw) ranging from 150 to 1,000,000.

31. A composition according to claim 1, wherein the at least one compound Y has a weight-average molecular mass (Mw) ranging from 200 to 1,000,000.

32. A composition according to claim 1, wherein the at least one compound X is present in an amount ranging from 0.5% to 95% by weight relative to the total weight of the composition.

33. A composition according to claim 1, wherein the at least one compound Y is present in an amount ranging from 0.05% to 95% by weight relative to the total weight of the composition.

34. A composition according to claim 1, wherein compounds X and Y are present in the composition(s) in a ratio X/Y ranging from 0.05 to 20.

35. A composition according to claim 1, wherein the at least one fluorescent dye absorbs in the visible radiation range at between 400 and 800 nm and re-emits light in the visible range at a higher wavelength.

36. A composition according to claim 35, wherein the at least one fluorescent dye is chosen from:
the methine derivatives of formula: 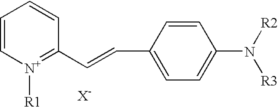
wherein R₁, R₂ and R₃, independently of each other, are chosen from hydrogen atoms; halogen atoms; C₆-C₃₀ aryl groups; hydroxyl groups; cyano groups; nitro groups; sulfo groups; amino groups; acylamino groups; di(C₁-C₆)alkylamino groups; dihydroxy(C₁-C₆)alkylamino groups; (C₁-C₆)alkylhydroxy(C₁-C₆)alkylamino groups; (C₁-C₆)alkoxy groups; (C₁-C₆)alkoxycarbonyl groups; carboxy(C₁-C₆)alkoxy groups; piperidinosulfonyl groups; pyrrolidino groups; (C₁-C₆)alkylhalo(C₁-C₆)alkylamino groups; benzoyl(C₁-C₆)alkyl groups; vinyl groups; formyl groups; C₆-C₃₀ aryl radicals optionally substituted with at least one group chosen from hydroxyl groups, linear, branched or cyclic C₁-C₆ alkoxy groups, and linear, branched or cyclic alkyl groups comprising from 1 to 22 carbon atoms, optionally substituted with at least one group chosen from hydroxyl, amino and C₁-C₆ alkoxy groups; linear, branched or cyclic alkyl radicals comprising from 1 to 22 carbon atoms, optionally substituted with at least one group chosen from hydroxyl groups, amino groups, linear, branched or cyclic C₁-C₆ alkoxy groups, optionally substituted aryl groups, carboxyl groups, sulfo groups and halogen atoms, wherein the alkyl radical is optionally interrupted with at least one heteroatom;
wherein two of the substituents R₂ and R₃ may optionally form, with the carbon atoms to which they are attached, a C₆-C₃₀ aromatic or heterocyclic nucleus comprising in total from 5 to 30 ring members and from 1 to 5 heteroatoms; wherein the ring is fused or non-fused and optionally substituted with at least one group chosen from C₁-C₄ alkyl, (C₁-C₄)alkoxy(C₁-C₄)alkyl, amino, di(C₁-C₄)alkylamino, halogen, phenyl, carboxyl and tri(C₁-C₄)alkylammonio(C₁-C₄)alkyl groups, and
X⁻ is an organic or mineral anion;
a fluorescent dye having the following structure: 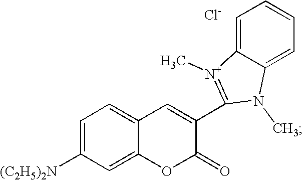
the methine hemicyanin having the following structure: 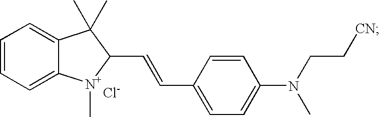
a fluorescent dye having the following structure: 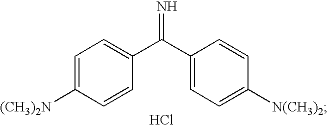
4,4′-(imidocarbonyl)bis(N,N-dimethylaniline) monohydrochloride having the following structure: 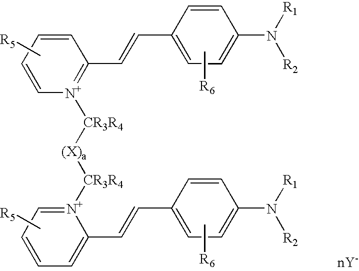
wherein:
R₁ and R₂, which may be identical or different, are chosen from:
hydrogen atoms;
linear or branched alkyl radicals comprising from 1 to 10 carbon atoms optionally interrupted and/or substituted with at least one heteroatom and/or group comprising at least one heteroatom and/or substituted with at least one halogen atom;
aryl or arylalkyl radicals, the aryl group comprising 6 carbon atoms and the alkyl radical comprising from 1 to 4 carbon atoms; the aryl radical optionally being substituted with at least one linear or branched alkyl radical comprising from 1 to 4 carbon atoms optionally interrupted and/or substituted with at least one heteroatom and/or group comprising at least one heteroatom and/or substituted with at least one halogen atom;
wherein R₁ and R₂ may optionally be linked so as to form a heterocycle with the nitrogen atom and may optionally comprise at least one other heteroatom, the heterocycle optionally being substituted with at least one linear or branched alkyl radical preferably comprising from 1 to 4 carbon atoms and optionally being interrupted and/or substituted with at least one heteroatom and/or group comprising at least one heteroatom and/or substituted with at least one halogen atom;
wherein R₁ or R₂ may optionally be engaged in a heterocycle comprising the nitrogen atom and one of the carbon atoms of the phenyl group bearing the nitrogen atom;
R₃ and R₄, which may be identical or different, are chosen from hydrogen atoms and alkyl radicals comprising from 1 to 4 carbon atoms;
R₅, which may be identical or different, is chosen from hydrogen atoms, halogen atoms and linear or branched alkyl radicals comprising from 1 to 4 carbon atoms, optionally interrupted with at least one heteroatom;
R₆, which may be identical or different, is chosen from hydrogen atoms; halogen atoms;
and linear or branched alkyl radicals comprising from 1 to 4 carbon atoms, optionally substituted and/or interrupted with at least one heteroatom and/or group bearing at least one heteroatom and/or substituted with at least one halogen atom;
X is chosen from:
linear or branched alkyl radicals comprising 1 to 14 carbon atoms or alkenyl radicals comprising from 2 to 14 carbon atoms, optionally interrupted and/or substituted with at least one heteroatom and/or group comprising at least one heteroatom and/or substituted with at least one halogen atom;
5- or 6-membered heterocyclic radicals optionally substituted with at least one linear or branched alkyl radical comprising from 1 to 14 carbon atoms, optionally substituted with at least one heteroatom; with at least one linear or branched aminoalkyl radical comprising from 1 to 4 carbon atoms, optionally substituted with at least one heteroatom; or with at least one halogen atom;
fused or non-fused aromatic or diaromatic radicals, optionally separated with at least one alkyl radical comprising from 1 to 4 carbon atoms, the aryl radical optionally being substituted with at least one halogen atom or with at least one alkyl radical comprising from 1 to 10 carbon atoms optionally substituted and/or interrupted with at least one heteroatom and/or group bearing at least one heteroatom;
dicarbonyl radicals; and
groups X optionally bearing at least one cationic charge;
a is equal to 0 or 1;
Y⁻, which may be identical or different, represents an organic or mineral anion;
n is an integer at least equal to 2 and at most equal to the number of cationic charges present in the fluorescent compound;
thioxanthene derivatives;
rhodamines;
stilbazolium dimers of formulae: 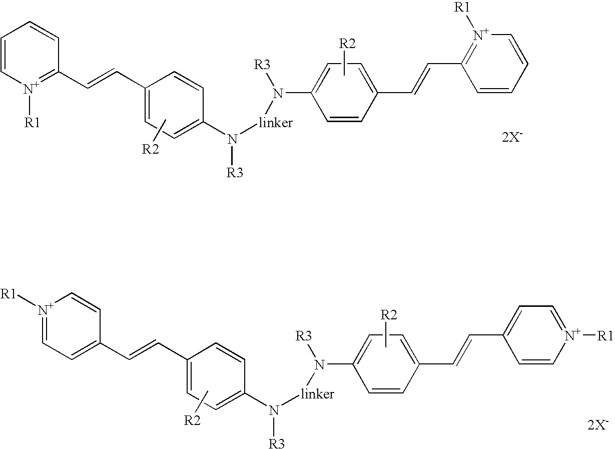
wherein:
R1, R2 and R3, independently of each other, are chosen from hydrogen atoms; halogen atoms; C6-C30 aryl groups; hydroxyl groups; cyano groups; nitro groups; sulfo groups; amino groups; acylamino groups; di(C1-C6)alkylamino groups; dihydroxy(C1-C6) alkylamino groups; (C1-C6)alkylhydroxy(C1-C6)alkylamino groups; (C1-C6)alkoxy groups; (C1-C6)alkoxycarbonyl groups; carboxy(C1-C6)alkoxy groups; piperidinosulfonyl groups; pyrrolidino groups; (C1-C6)alkylhalo(C1-C6)alkylamino groups; benzoyl(C1-C6)alkyl groups; vinyl groups; formyl groups; C6-C30 aryl radicals optionally substituted with at least one group chosen from hydroxyl groups, linear, branched or cyclic C1-C6 alkoxy groups, and linear, branched or cyclic alkyl groups comprising from 1 to 22 carbon atoms, optionally substituted with at least one group chosen from hydroxyl, amino and C1-C6 alkoxy groups; linear, branched or cyclic alkyl radicals comprising from 1 to 22 carbon atoms, optionally substituted with at least one group chosen from hydroxyl groups, amino groups, linear, branched or cyclic C1-C6 alkoxy groups, optionally substituted aryl groups, carboxyl groups, sulfo groups and halogen atoms, wherein the alkyl radical is optionally interrupted with at least one heteroatom;
the linker is chosen from saturated or unsaturated, aliphatic or alicyclic C1-C12 hydrocarbon-based chains, wherein at least one carbon atom of the hydrocarbon-based chain is optionally replaced with at least one oxygen atom and/or at least one group NR wherein R is chosen from hydrogen atoms and alkyl radicals, the hydrocarbon-based chain not comprising any diazo, nitro, nitroso or peroxide groups, and the hydrocarbon-based chain not ending at either of its ends with a heteroatom; and
X⁻ represents an anionic counterion;
the methine trimers and tetramers of formulae: 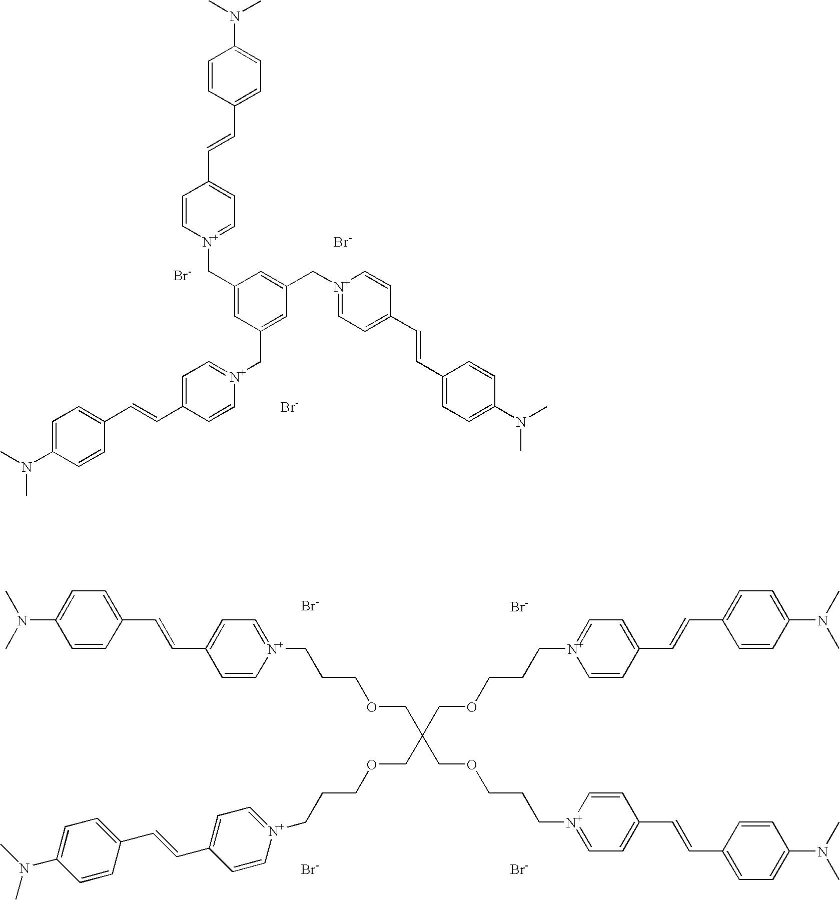
xanthene derivatives of formula: 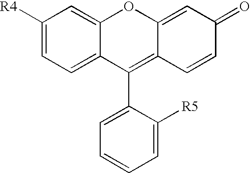
wherein R4 and R5, independently of each other, are chosen from hydrogen atoms; halogen atoms; C6-C30 aryl groups; hydroxyl groups; cyano groups; nitro groups; sulfo groups; amino groups; acylamino groups; di(C1-C6)alkylamino groups; dihydroxy(C1-C6) alkylamino groups; (C1-C6)alkylhydroxy(C1-C6)alkylamino groups; (C1-C6)alkoxy groups; (C1-C6)alkoxycarbonyl groups; carboxy(C1-C6)alkoxy groups; piperidinosulfonyl groups; pyrrolidino groups; (C1-C6)alkylhalo(C1-C6)alkylamino groups; benzoyl(C1-C6)alkyl groups; vinyl groups; formyl groups; C6-C30 aryl radicals optionally substituted with at least one group chosen from hydroxyl groups, linear, branched or cyclic C1-C6 alkoxy groups, and linear, branched or cyclic alkyl groups comprising from 1 to 22 carbon atoms, optionally substituted with at least one group chosen from hydroxyl, amino and C1-C6 alkoxy groups; and linear, branched or cyclic alkyl radicals comprising from 1 to 22 carbon atoms, optionally substituted with at least one group chosen from hydroxyl groups, amino groups, linear, branched or cyclic C1-C6 alkoxy groups, optionally substituted aryl groups, carboxyl groups, sulfo groups and halogen atoms, wherein the alkyl radical is optionally interrupted with a heteroatom;
the naphthalimides of formula: 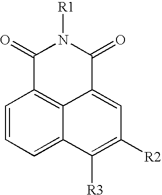
wherein R1, R2 and R3, independently of each other, are chosen from hydrogen atoms; halogen atoms; C6-C30 aryl groups; hydroxyl groups; cyano groups; nitro groups; sulfo groups; amino groups; acylamino groups; di(C1-C6)alkylamino groups; dihydroxy(C1-C6) alkylamino groups; (C1-C6)alkylhydroxy(C1-C6)alkylamino groups; (C1-C6)alkoxy groups; (C1-C6)alkoxycarbonyl groups; carboxy(C1-C6)alkoxy groups; piperidinosulfonyl groups; pyrrolidino groups; (C1-C6)alkylhalo(C1-C6)alkylamino groups; benzoyl(C1-C6)alkyl groups; vinyl groups; formyl groups; C6-C30 aryl radicals optionally substituted with at least one group chosen from hydroxyl groups, linear, branched or cyclic C1-C6 alkoxy groups, and linear, branched or cyclic alkyl groups comprising from 1 to 22 carbon atoms, optionally substituted with at least one group chosen from hydroxyl, amino and C1-C6 alkoxy groups; linear, branched or cyclic alkyl radicals comprising from 1 to 22 carbon atoms, optionally substituted with at least one group chosen from hydroxyl groups, amino groups, linear, branched or cyclic C1-C6 alkoxy groups, optionally substituted aryl groups, carboxyl groups, sulfo groups and halogen atoms, wherein the alkyl radical is optionally interrupted with at least one heteroatom; wherein the substituents R1, R2 and R3 may form, with the carbon atoms to which they are attached, a C6-C30 aromatic or non-aromatic or heterocyclic ring comprising in total from 5 to 30 ring members and from 1 to 5 heteroatoms; these rings being fused or non-fused, optionally comprising a carbonyl group, and being unsubstituted or substituted with at least one group chosen from C1-C4 alkyl, (C1-C4)alkoxy(C1-C4)alkyl, amino, di(C1-C4)alkylamino, halogen, phenyl, carboxyl and tri(C1-C4)alkylammonio(C1-C4)alkyl groups;
diketopyrrolopyrroles of formula: 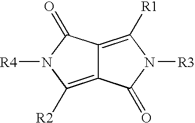
wherein R1, R2, R3 and R4, independently of each other, represent hydrogen atoms; halogen atoms; C6-C30 aryl groups; hydroxyl groups; cyano groups; nitro groups; sulfo groups; amino groups; acylamino groups; di(C1-C6)alkylamino groups; dihydroxy(C1-C6) alkylamino groups; (C1-C6)alkylhydroxy(C1-C6)alkylamino groups; (C1-C6)alkoxy groups; (C1-C6)alkoxycarbonyl groups; carboxy(C1-C6)alkoxy groups; piperidinosulfonyl groups; pyrrolidino groups; (C1-C6)alkylhalo(C1-C6)alkylamino groups; benzoyl(C1-C6)alkyl groups; vinyl groups; formyl groups; C6-C30 aryl radicals optionally substituted with at least one group chosen from hydroxyl groups, linear, branched or cyclic C1-C6 alkoxy groups, and linear, branched or cyclic alkyl groups comprising from 1 to 22 carbon atoms, optionally substituted with at least one group chosen from hydroxyl, amino and C1-C6 alkoxy groups; and linear, branched or cyclic alkyl radicals comprising from 1 to 22 carbon atoms, optionally substituted with at least one group chosen from hydroxyl groups, amino groups, linear, branched or cyclic C1-C6 alkoxy groups, optionally substituted aryl groups, carboxyl groups, sulfo groups and halogen atoms, wherein the alkyl radical is optionally interrupted with at least one heteroatom;
the dicyanopyrazine derivatives of formulae: 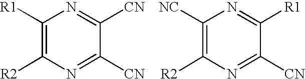
wherein R1 and R2, independently of each other, are chosen from hydrogen atoms; halogen atoms; C6-C30 aryl groups; hydroxyl groups; cyano groups; nitro groups; sulfo groups; amino groups; acylamino groups; di(C1-C6)alkylamino groups; dihydroxy(C1-C6) alkylamino groups; (C1-C6)alkylhydroxy(C1-C6)alkylamino groups; (C1-C6)alkoxy groups; (C1-C6)alkoxycarbonyl groups; carboxy(C1-C6)alkoxy groups; piperidinosulfonyl groups; pyrrolidino groups; (C1-C6)alkylhalo(C1-C6)alkylamino groups; benzoyl(C1-C6)alkyl groups; vinyl groups; formyl groups; C6-C30 aryl radicals optionally substituted with at least one group chosen from hydroxyl groups, linear, branched or cyclic C1-C6 alkoxy groups, and linear, branched or cyclic alkyl groups comprising from 1 to 22 carbon atoms, optionally substituted with at least one group chosen from hydroxyl, amino and C1-C6 alkoxy groups; and linear, branched or cyclic alkyl radicals comprising from 1 to 22 carbon atoms, optionally substituted with at least one group chosen from hydroxyl groups, amino groups, linear, branched or cyclic C1-C6 alkoxy groups, optionally substituted aryl groups, carboxyl groups, sulfo groups and halogen atoms, wherein the alkyl radical is optionally interrupted with at least one heteroatom;
wherein two of the substituents R1 and R2 may optionally form, with the carbon atoms to which they are attached, a C6-C30 aromatic or non-aromatic or heterocyclic ring comprising in total from 5 to 30 ring members and from 1 to 5 heteroatoms; these rings being fused or non-fused, optionally comprising a carbonyl group, and being unsubstituted or substituted with at least one group chosen from C1-C4 alkyl, (C1-C4)alkoxy(C1-C4)alkyl, amino, di(C1-C4)alkylamino, halogen, phenyl, carboxyl, and tri(C1-C4)alkylammonio(C1-C4)alkyl groups;
wherein two of the substituents R2 and R3 may optionally form, with the carbon atoms to which they are attached, a C6-C30 aromatic or heterocyclic nucleus comprising in total from 5 to 30 ring members and from 1 to 5 heteroatoms; the ring being fused or non-fused, wherein the ring and/or the fused ring are unsubstituted or substituted with at least one group chosen from C1-C4 alkyl, (C1-C4)alkoxy(C1-C4)alkyl, amino, di(C1-C4)alkylamino, halogen, phenyl, carboxyl and tri(C1-C4)alkylammonio(C1-C4)alkyl groups;
coumarin derivatives of the following formulae: 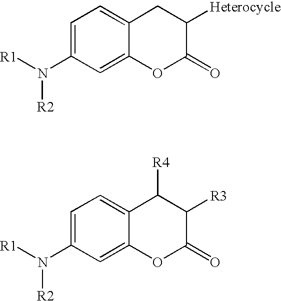
wherein the heterocycle is chosen from furan, thiophene, 2H-pyrrole, 2-pyrroline, pyrrolidine, 1,3-dioxolane, oxazole, thiazole, imidazole, 2-imidazoline, imidazoline, pyrazole, 2-pyrazoline, pyrazolidine, isoxazole, isothiazole, 1,2,3-oxadiazole, 1,2,3-triazole, 1,3,4-thiadiazole, 2H-pyran, 4H-pyran, pyridine, piperidine, 1,4-dioxane, morpholine, 1,4-dithiane, thiomorpholine, pyradizine, pyrimidine, pyrazine, piperazine, 1,3,5-triazine, 1,3,5-trithiane, indolizine, indole, isoindole, 3H-indole, indoline, benzo[b]furan, benzo[b]thiophene, 1H-indazole, benzimidazole, benzothiazole, purine, 4H-quinolizine, quinoline, isoquinoline, cinnoline, phthalazine, quinazoline, quinoxaline, 1,8-naphthyridine, pteridine, quinuclidine, carbazole, acridine, phenazine, phenothiazine, phenoxazine, indene, naphthalene, azulene, fluorene, anthracene, norbornane, and adamantane; and wherein R1, R2, R3 and R4, independently of each other, are chosen from hydrogen atoms; halogen atoms; C6-C30 aryl groups; hydroxyl groups; cyano groups; nitro groups; sulfo groups; amino groups; acylamino groups; di(C1-C6)alkylamino groups; dihydroxy(C1-C6) alkylamino groups; (C1-C6)alkylhydroxy(C1-C6)alkylamino groups; (C1-C6)alkoxy groups; (C1-C6)alkoxycarbonyl groups; carboxy(C1-C6)alkoxy groups; piperidinosulfonyl groups; pyrrolidino groups; (C1-C6)alkylhalo(C1-C6)alkylamino groups; benzoyl-(C1-C6)alkyl groups; vinyl groups; formyl groups; C6-C30 aryl radicals optionally substituted with at least one group chosen from hydroxyl groups, linear, branched or cyclic C1-C6 alkoxy groups, and linear, branched or cyclic alkyl groups comprising from 1 to 22 carbon atoms, optionally substituted with at least one group chosen from hydroxyl, amino and C1-C6 alkoxy groups; and linear, branched or cyclic alkyl radicals comprising from 1 to 22 carbon atoms, optionally substituted with at least one group chosen from hydroxyl groups, amino groups, linear, branched or cyclic C1-C6 alkoxy groups, optionally substituted aryl groups, carboxyl groups, sulfo groups and halogen atoms, wherein the alkyl radical is optionally interrupted with at least one heteroatom; wherein the substituents R1, R2, R3 and R4 may optionally form, with the carbon atoms to which they are attached, a C6-C30 aromatic or non-aromatic or heterocyclic ring comprising in total from 5 to 30 ring members and from 1 to 5 heteroatoms; wherein the ring is fused or non-fused, optionally comprising a carbonyl group, and being unsubstituted or substituted with at least one group chosen from C1-C4 alkyl, (C1-C4)alkoxy(C1-C4)alkyl, amino, di(C1-C4)alkylamino, halogen, phenyl, carboxyl and tri(C1-C4)alkylammonio(C1-C4)alkyl groups;
wherein two of the substituents R3 and R4 may optionally form, with the carbon atoms to which they are attached, a C6-C30 aromatic or heterocyclic nucleus comprising in total from 5 to 30 ring members and from 1 to 5 heteroatoms; wherein the ring is fused or non-fused, and wherein the ring and/or the possible fused ring are unsubstituted or substituted with at least one group chosen from C1-C4 alkyl, (C1-C4)alkoxy(C1-C4)alkyl, amino, di(C1-C4)alkylamino, halogen, phenyl, carboxyl and tri(C1-C4)alkylammonio(C1-C4)alkyl groups;
naphtholactam derivatives: 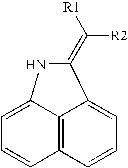
wherein R1 and R2 are defined as above;
azlactone derivatives: 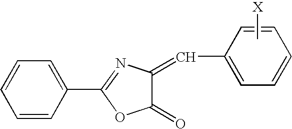
wherein X has the same definition as R1 described above;
methine derivatives of formula: 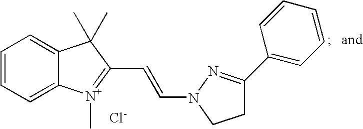
and
oxazine and thiazine derivatives of formula: 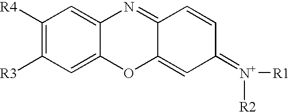
wherein R1, R2, R3 and R4 are as defined above.

37. A composition according to claim 1, further comprising at least one optical brightener.

38. A composition according to claim 37, wherein the at least one optical brightener is chosen from:
stilbene derivatives of naphthotriazole, disodium 4,4′-distyrylbiphenyl sulfonate, cationic derivatives of aminocoumarin, diethylaminomethylcoumarin, 4-methyl-7-diethylcoumarin, sodium 4,4′-bis[(4,6-dianilino-1,3,5-triazin-2-yl)amino]stilbene-2,2′-disulfonate, 4,4′-bis-[(4-anilino-6-bis(2-hydroxyethyl)amino-1,3,5-triazin-2-yl)amino]stilbene-2,2′-disulfonic acid, 4,4′-bis[anilino-6-morpholine-1,3,5-triazin-2-yl)amino]stilbene, and disodium 4,4′-bis[(4-anilino-6-(2-hydroxyethyl)methylamino-1,3,5-triazin-2-yl)-amino]stilbene-2,2′-sulfonate;
2,5-thiophenediylbis(5-tert-butyl-1,3-benzoxazole);
anionic derivatives of diaminostilbene;
optical brightener lakes;
naphthalimides;
1,4-distyrylbenzene derivatives of formula: 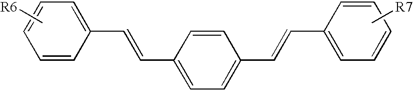
wherein R6 and R7, independently of each other, are chosen from hydrogen atoms; halogen atoms; C6-C30 aryl groups; hydroxyl groups; cyano groups; nitro groups; sulfo groups; amino groups; acylamino groups; di(C1-C6)alkylamino groups; dihydroxy(C1-C6)alkylamino groups; (C1-C6)alkylhydroxy(C1-C6)alkylamino groups; (C1-C6)alkoxy groups; (C1-C6)alkoxycarbonyl groups; carboxy(C1-C6)alkoxy groups; piperidinosulfonyl groups; pyrrolidino groups; (C1-C6)alkylhalo(C1-C6)alkylamino groups; benzoyl(C1-C6)alkyl groups; vinyl groups; formyl groups; C6-C30 aryl radicals optionally substituted with at least one group chosen from hydroxyl groups, linear, branched or cyclic C1-C6 alkoxy groups, and linear, branched or cyclic alkyl groups comprising from 1 to 22 carbon atoms, optionally substituted with at least one group chosen from hydroxyl, amino and C1-C6 alkoxy groups; and linear, branched or cyclic alkyl radicals comprising from 1 to 22 carbon atoms, optionally substituted with at least one group chosen from hydroxyl groups, amino groups, linear, branched or cyclic C1-C6 alkoxy groups, optionally substituted aryl groups, carboxyl groups, sulfo groups and halogen atoms, wherein the alkyl radical is optionally interrupted with at least one heteroatom;
the 4,4′-distyrylbiphenyl derivatives of formula: 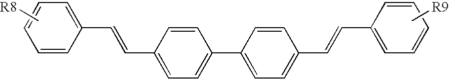
wherein R8 and R9, independently of each other, are chosen from hydrogen atoms; halogen atoms; C6-C30 aryl groups; hydroxyl groups; cyano groups; nitro groups; sulfo groups; amino groups; acylamino groups; di(C1-C6)alkylamino groups; dihydroxy(C1-C6)alkylamino groups; (C1-C6)alkylhydroxy(C1-C6)alkylamino groups; (C1-C6)alkoxy groups; (C1-C6)alkoxycarbonyl groups; carboxy(C1-C6)alkoxy groups; piperidinosulfonyl groups; pyrrolidino groups; (C1-C6)alkylhalo(C1-C6)alkylamino groups; benzoyl(C1-C6)alkyl groups; vinyl groups; formyl groups; C6-C30 aryl radicals optionally substituted with at least one group chosen from hydroxyl groups, linear, branched or cyclic C1-C6 alkoxy groups, and linear, branched or cyclic alkyl groups comprising from 1 to 22 carbon atoms, optionally substituted with at least one hydroxyl, amino or C1-C6 alkoxy groups; linear, branched or cyclic alkyl radicals comprising from 1 to 22 carbon atoms, optionally substituted with at least one group chosen from hydroxyl groups, amino groups, linear, branched or cyclic C1-C6 alkoxy groups, optionally substituted aryl groups, carboxyl groups, sulfo groups and halogen atoms, wherein the alkyl radical is optionally interrupted with at least one heteroatom;
the triazinylaminostilbene derivatives of formula: 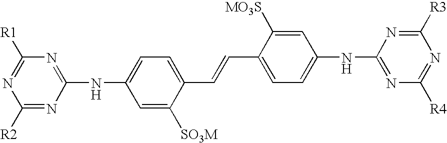
wherein R1, R2, R3 and R4, independently of each other, are chosen from hydrogen atoms; halogen atoms; C6-C30 aryl groups; hydroxyl groups; cyano groups; nitro groups; sulfo groups; amino groups; acylamino groups; di(C1-C6)alkylamino groups; dihydroxy(C1-C6)alkylamino groups; (C1-C6)alkylhydroxy(C1-C6)alkylamino groups; (C1-C6)alkoxy groups; (C1-C6)alkoxycarbonyl groups; carboxy(C1-C6)alkoxy groups; piperidinosulfonyl groups; pyrrolidino groups; (C1-C6)alkylhalo(C1-C6)alkylamino groups; benzoyl-(C1-C6)alkyl groups; vinyl groups; formyl groups; C6-C30 aryl radicals optionally substituted with at least one group chosen from hydroxyl groups, linear, branched or cyclic C1-C6 alkoxy groups, and linear, branched or cyclic alkyl groups comprising from 1 to 22 carbon atoms, optionally substituted with at least one hydroxyl, amino or C1-C6 alkoxy group; and linear, branched or cyclic alkyl radicals comprising from 1 to 22 carbon atoms, optionally substituted with at least one group chosen from hydroxyl groups, amino groups, linear, branched or cyclic C1-C6 alkoxy groups, optionally substituted aryl groups, carboxyl groups, sulfo groups and halogen atoms, wherein the alkyl radical is optionally interrupted with at least one heteroatom;
and M is a monovalent or divalent cation derived from the family of alkali metals or alkaline-earth metals;
the bis(benzoxazole) derivatives of formula: 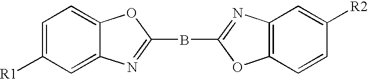
wherein R1 and R2, independently of each other, are chosen from hydrogen atoms; halogen atoms; C6-C30 aryl groups; hydroxyl groups; cyano groups; nitro groups; sulfo groups; amino groups; acylamino groups; di(C1-C6)alkylamino groups; dihydroxy(C1-C6)alkylamino groups; (C1-C6)alkylhydroxy(C1-C6)alkylamino groups; (C1-C6)alkoxy groups; (C1-C6)alkoxycarbonyl groups; carboxy(C1-C6)alkoxy groups; piperidinosulfonyl groups; pyrrolidino groups; (C1-C6)alkylhalo(C1-C6)alkylamino groups; benzoyl-(C1-C6)alkyl groups; vinyl groups; formyl groups; C6-C30 aryl radicals optionally substituted with at least one group chosen from hydroxyl groups, linear, branched or cyclic C1-C6 alkoxy groups, and linear, branched or cyclic alkyl groups comprising from 1 to 22 carbon atoms, optionally substituted with at least one hydroxyl, amino or C1-C6 alkoxy group; and linear, branched or cyclic alkyl radicals comprising from 1 to 22 carbon atoms, optionally substituted with at least one group chosen from hydroxyl groups, amino groups, linear, branched or cyclic C1-C6 alkoxy groups, optionally substituted aryl groups, carboxyl groups, sulfo groups and halogen atoms, wherein the alkyl radical is optionally interrupted with at least one heteroatom; and
B is chosen from: 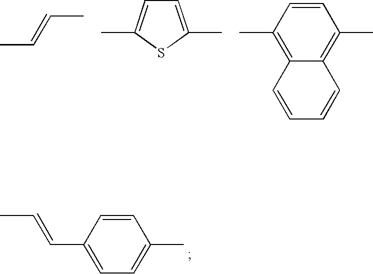
cationic or non-cationic bis(benzimidazoles); and
anionic or non-anionic 1,3-diphenyl-2-pyrazolines.

39. A composition according to claim 37, wherein the at least one fluorescent dye and, if present, the at least one optical brightener is present in an amount ranging from 0.001% to 40% by weight relative to the total weight of the composition.

40. A composition according to claim 1, comprising at least one organic solvent chosen from organic oils; silicones; mineral oils; plant oils; C₅-C₂₀ alkanes; acetone; methyl ethyl ketone; esters of liquid C₁-C₂₀ acids and of C₁-C₈ alcohols; dimethoxyethane; diethoxyethane; liquid C₁₀-C₃₀ fatty alcohols; esters of fatty alcohols or of fatty acids; polybutene oil; isononyl isononanoate; isostearyl malate; pentaerythrityl tetraisostearate; tridecyl trimellitate; and mixtures thereof.

41. A composition according to the claim 40, wherein the at least one organic solvent is chosen from silicones.

42. A process for dyeing keratin fibers, comprising
applying to the fibers at least one compound X, at least one compound Y and at least one fluorescent dye, wherein at least one of compounds X or Y is a silicone compound, and wherein when the compounds X and Y are placed in contact with each other, they react together via
a hydrosilylation reaction optionally in the presence of at least one hydrosilylation catalyst;
a condensation reaction; or
a crosslinking reaction in the presence of at least one peroxide.

43. A multi-compartment device comprising at least two compositions (A) and (B) packaged separately, the kit comprising i) at least one compound X, ii) at least one compound Y, wherein at least one of composition (A) and/or (B) comprises at least one fluorescent dye; and iii) optionally at least one catalyst or one peroxide; wherein the catalyst, when it is present, and the peroxide are not simultaneously present in the same composition; the compounds X and Y, when placed in contact with each other, react together via a hydrosilylation reaction, or via a condensation reaction, or via a crosslinking reaction in the presence of at least one peroxide.

44. A process for lightening of dark keratin fibers, comprising applying to dark keratin fibers a composition comprising:
i) at least one compound X;
ii) at least one compound Y;
iii) at least one fluorescent dye;
iv) optionally at least one optical brightener;
v) optionally at least one organic solvent chosen from organic oils; silicones; mineral oils; plant oils; C₅-C₂₀ alkanes; acetone; methyl ethyl ketone; esters of liquid C₁-C₂₀ acids and of C₁-C₈ alcohols; dimethoxyethane; diethoxyethane; liquid C₁₀-C₃₀ fatty alcohols; esters of fatty alcohols or of fatty acids; polybutene oil; isononyl isononanoate; isostearyl malate; pentaerythrityl tetraisostearate; tridecyl trimellitate; and mixtures thereof, wherein at least one of compounds X or Y is a silicone compound, and wherein the compounds X and Y, when they are placed in contact with each other, react together via:
a hydrosilylation reaction optionally in the presence of at least one hydrosilylation catalyst;
a condensation reaction; or
a crosslinking reaction in the presence of at least one peroxide.
